SEQUENCE LISTING (26 sequences)

SEQ ID NO: 1 - protein, 120 aa, Artificial Sequence
  Full VH Sequence of 17E7
EVQLQQSGTVLARPGASVKMSCKTSGYSFTTYWMHWVKQRPGQGLEWIGAIYPGNSDTYYSQKFKDKAKLTAVTSASTAYMELSSLTNEDSAVYYCTRSGGYDVGGLDYWGQGTTLTVSS

SEQ ID NO: 2 - protein, 5 aa, Artificial Sequence
  CDR-H1 of 17E7
TYWMH

SEQ ID NO: 3 - protein, 17 aa, Artificial Sequence
  CDR-H2 of 17E7
AIYPGNSDTYYSQKFKD

SEQ ID NO: 4 - protein, 11 aa, Artificial Sequence
  CDR-H3 of 17E7
SGGYDVGGLDY

SEQ ID NO: 5 - protein, 107 aa, Artificial Sequence
  Full VL Sequence of 17E7
DIVMTQSPATLSVTPGDGVSLSCRASQSISDYLHWYQQKSHESPRLLIKFASQSISGIPSRFSGSGSGSDFTLSINSVEPEDVGMYYCQNGHSFPFTFGSGTKLELK

SEQ ID NO: 6 - protein, 11 aa, Artificial Sequence
  CDR-L1 of 17E7
RASQSISDYLH

SEQ ID NO: 7 - protein, 7 aa, Artificial Sequence
  CDR-L2 of 17E7
FASQSIS

SEQ ID NO: 8 - protein, 9 aa, Artificial Sequence
  CDR-L3 of 17E7
QNGHSFPFT

SEQ ID NO: 9 - protein, 842 aa, Homo sapiens
MKVISLFILVGFIGEFQSFSSASSPVNCQWDFYAPWSECNGCTKTQTRRRSVAVYGQYGQPCVGNAFETQSCEPTRGCPTEEGCGERFRCFSGQCISKSLVCNGDSDCDEDSADEDRCEDSERRPSCDIDKPPPNIELTGNGYNELTGQFRNRVINTKSFGGQCRKVFSGDGKDFYRLSGNVLSYTFQVKINNDFNYEFYNSTWSYVKHTSTEHTSSSRKRSFFRSSSSSSRSYTSHTNEIHKGKSYQLLVVENTVEVAQFINNNPEFLQLAEPFWKELSHLPSLYDYSAYRRLIDQYGTHYLQSGSLGGEYRVLFYVDSEKLKQNDFNSVEEKKCKSSGWHFVVKFSSHGCKELENALKAASGTQNNVLRGEPFIRGGGAGFISGLSYLELDNPAGNKRRYSAWAESVTNLPQVIKQKLTPLYELVKEVPCASVKKLYLKWALEEYLDEFDPCHCRPCQNGGLATVEGTHCLCHCKPYTFGAACEQGVLVGNQAGGVDGGWSCWSSWSPCVQGKKTRSRECNNPPPSGGGRSCVGETTESTQCEDEELEHLRLLEPHCFPLSLVPTEFCPSPPALKDGFVQDEGTMFPVGKNVVYTCNEGYSLIGNPVARCGEDLRWLVGEMHCQKIACVLPVLMDGIQSHPQKPFYTVGEKVTVSCSGGMSLEGPSAFLCGSSLKWSPEMKNARCVQKENPLTQAVPKCQRWEKLQNSRCVCKMPYECGPSLDVCAQDERSKRILPLTVCKMHVLHCQGRNYTLTGRDSCTLPASAEKACGACPLWGKCDAESSKCVCREASECEEEGFSICVEVNGKEQTMSECEAGALRCRGQSISVTSIRPCAAETQ

SEQ ID NO: 10 - protein, 150 aa, Homo sapiens
PLTQAVPKCQRWEKLQNSRCVCKMPYECGPSLDVCAQDERSKRILPLTVCKMHVLHCQGRNYTLTGRDSCTLPASAEKACGACPLWGKCDAESSKCVCREASECEEEGFSICVEVNGKEQTMSECEAGALRCRGQSISVTSIRPCAAETQ

SEQ ID NO: 11 - protein, 119 aa, Artificial Sequence
  Full VH Sequence of 2H2
QVQLQQPGAELMKPGASVKLSCKASGYIFTSYWMHWVKQRPGRGLEWIGRIDPNSGGTNYNEKFKSKATLTVDKPSSTVYMKISSLTSEDSAVYYCAREHDYDYAMDYWGQGTSVTVSS

SEQ ID NO: 12 - protein, 5 aa, Artificial Sequence
  CDR-H1 of 2H2
SYWMH

SEQ ID NO: 13 - protein, 17 aa, Artificial Sequence
  CDR-H2 of 2H2
RIDPNSGGTNYNEKFKS

SEQ ID NO: 14 - protein, 10 aa, Artificial Sequence
  CDR-H3 of 2H2
EHDYDYAMDY

SEQ ID NO: 15 - protein, 112 aa, Artificial Sequence
  Full VL Sequence of 2H2
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGSGTKLEIK

SEQ ID NO: 16 - protein, 16 aa, Artificial Sequence
  CDR-L1 of 2H2
RSSKSLLHSNGNTYLY

SEQ ID NO: 17 - protein, 7 aa, Artificial Sequence
  CDR-L2 of 2H2
RMSNLAS

SEQ ID NO: 18 - protein, 9 aa, Artificial Sequence
  CDR-L3 of 2H2
MQHLEYPFT

SEQ ID NO: 19 - protein, 115 aa, Artificial Sequence
  Full VH Sequence of 73D1
EVQLQQSGPELVKPGASVKMSCKASGYTFTDYNMHWVKQSHGKSLEWIGYINPNNGGPSYNQNFKGKATLTVNKSSSTAYMELRSLTSEDSAVYYCACGNYPDYWGQGTTLTVSS

SEQ ID NO: 20 - protein, 5 aa, Artificial Sequence
  CDR-H1 of 73D1
DYNMH

SEQ ID NO: 21 - protein, 17 aa, Artificial Sequence
  CDR-H2 of 73D1
YINPNNGGPSYNQNFKG

SEQ ID NO: 22 - protein, 6 aa, Artificial Sequence
  CDR-H3 of 73D1
GNYPDY

SEQ ID NO: 23 - protein, 107 aa, Artificial Sequence
  Full VL Sequence of 73D1
DIQMTQSPASLSASVGETVTITCRASGNIHNYLAWYQQKQGKSPQLLVYNAKTLADGVPSRFSGSGSGTQYSLKINSLQPEDFGSYYCQHFWSTPLTFGAGTKLELK

SEQ ID NO: 24 - protein, 11 aa, Artificial Sequence
  CDR-L1 of 73D1
RASGNIHNYLA

SEQ ID NO: 25 - protein, 7 aa, Artificial Sequence
  CDR-L2 of 73D1
NAKTLAD

SEQ ID NO: 26 - protein, 9 aa, Artificial Sequence
  CDR-L3 of 73D1
QHFWSTPLT

(19) United States
(12) Patent Application Publication
(10) Pub. No.: US 2022/0380444 A1
(43) Pub. Date: Dec. 1, 2022

(54) Anti-C7 Antibody Or Antibody Fragment

(71) Applicant: University College Cardiff Consultants Ltd, Cardiff (GB)

(72) Inventors: Bryan Paul Morgan, Cardiff (GB); Wioleta Milena Zelek, Cardiff (GB)

(21) Appl. No.: 17/636,425

(22) PCT Filed: Aug. 20, 2020
(86) PCT No.: PCT/EP2020/073430

§ 371 Date: Feb. 18, 2022

(30) Foreign Application Priority Data
Aug. 20, 2019 (GB) 1911931.2

(51) Int. Cl. C07K 16/18

(52) U.S. Cl. CPC C07K 16/18; C07K 2317/51; C07K 2317/515; C07K 2317/92; C07K 2317/24

(57) ABSTRACT

An antibody or antibody fragment that binds to complement protein C7 in isolation or as part of a protein complex and the various uses, for example for the treatment of a disease associated with the dysregulation of complement in a subject.

### TECHNICAL FIELD

The present invention relates generally to antibodies and antibody fragments that bind to the complement protein C7 in isolation or as part of a protein complex and inhibit complement activation. The present invention further relates to nucleic acid sequences encoding said antibodies or antibody fragments, cells comprising said nucleic acid sequences, and cells secreting said antibodies or antibody fragments. In particular embodiments, the present invention relates to the hybridoma deposited at the European Collection of Authenticated Cell Cultures (ECACC-Culture Collections, Public Health England, Porton Down, Salisbury, Wiltshire, SP4 OJG, United Kingdom) on 16 Apr. 2019 and having accession number 19041601, the hybridoma deposited at ECACC on 6 Aug. 2019 and having accession number 19080602, the hybridoma deposited at ECACC on 6 Aug. 2019 having accession number 19080601, the hybridoma deposited at ECACC on 6 Aug. 2019 having accession number 19080603, and antibodies obtained from or obtainable from said hybridomas. The present invention also relates to the use of the antibodies and antibody fragments described herein for treating or preventing a disease associated with the dysregulation of complement in a subject.

### BACKGROUND

Complement is a key part of the innate immune system, on the front line in defence of the body from invading pathogens. This system has evolved to protect the body from foreign organisms, however, dysregulation of complement can lead to pathology, for example involved in inflammatory and degenerative diseases. There are only three anti-complement drugs approved by the Food and Drug Administration (FDA). The anti-C5 monoclonal antibodies Eculizumab and Ravulizumab are approved for treating paroxysmal nocturnal hemoglobinuria (PNH) and atypical haemolytic uremic syndrome (aHUS). A Cl inhibitor, marketed under several names including Cinryze®, is approved for treating hereditary angioedema (HAE).

It is therefore desirable to provide new and improved drugs for targeting complement to treat or prevent pathologies associated with the dysregulation of complement.

### SUMMARY

In accordance with a first aspect of the present invention there is provided an antibody or antibody fragment that binds to the complement protein C7 in isolation or as part of a protein complex and inhibits complement activation.

In a second aspect of the present invention there is provided an antibody obtained from or obtainable from the hybridoma having the ECACC accession number 19041601 or a humanized or affinity-matured version of the antibody obtained from or obtainable from the hybridoma having the ECACC accession number 19041601.

In a third aspect of the present invention there is provided an antibody obtained from or obtainable from the hybridoma having the ECACC accession number 19080602 or a humanized or affinity-matured version of the antibody obtained from or obtainable from the hybridoma having the ECACC accession number 19080602.

In a fourth aspect of the present invention there is provided an antibody obtained from or obtainable from the hybridoma having the ECACC accession number 19080601 or a humanized or affinity-matured version of the antibody obtained from or obtainable from the hybridoma having the ECACC accession number 19080601.

In a fifth aspect of the present invention there is provided an antibody obtained from or obtainable from the hybridoma having the ECACC accession number 19080603 or a humanized or affinity-matured version of the antibody obtained from or obtainable from the hybridoma having the ECACC accession number 19080603.

In accordance with a sixth aspect of the present invention there is provided a pharmaceutical composition comprising an antibody or antibody fragment according to the first or second or third or fourth or fifth aspect of the present invention and a pharmaceutically acceptable excipient, diluent, or carrier. The pharmaceutical composition may, for example, comprise more than one antibody or antibody fragment according to the first or second aspect of the present invention, which may be the same or different. The antibodies or antibody fragments may, for example, be monoclonal antibodies or antibody fragments.

In accordance with a seventh aspect of the present invention there is provided a nucleic acid sequence encoding an antibody or antibody fragment according to the first or second or third or fourth or fifth aspect of the present invention.

In accordance with a eighth aspect of the present invention there is provided a cell comprising a nucleic acid sequence according to the seventh aspect of the present invention. In accordance with a ninth aspect of the present invention there is provided a cell secreting an antibody or antibody fragment according to the first or second or third or fourth or fifth aspect of the present invention. The cell may, for example, be an established cell line. The cell may, for example, be the hybridoma having the ECACC accession number 19041601. The cell may, for example, be the hybridoma having the ECACC accession number 19080601. The cell may, for example, be the hybridoma having the ECACC accession number 19080602. The cell may, for example, be the hybridoma having the ECACC accession number 19080603.

In accordance with a tenth aspect of the present invention there is provided an antibody, antibody fragment, or pharmaceutical composition according to any aspect of the present invention for use in the treatment or prevention of a disease associated with the dysregulation of complement.

In accordance with an eleventh aspect of the present invention there is provided a method of treating or preventing a disease associated with the dysregulation of complement in a subject, the method comprising administering an antibody or antibody fragment or pharmaceutical composition according to any aspect of the present invention to the subject.

Certain embodiments of any aspect of the present invention may provide one or more of the following advantages:
new target protein;
new target epitope on C7 (in isolation or as part of a protein complex, for example as part of the C5b67 complex);
new mechanism of action (e.g. blocking formation of the membrane attack complex (MAC) downstream of C5 and C6);
good binding affinity (KD) and/or binding association rate (ka) and/or binding dissociation rate (kd) for C7 in isolation or as part of a protein complex (e.g. similar to that of the clinically available antibody Eculizumab for C5);
good serum complement inhibition levels (e.g. inhibition in vitro at similar levels to that of the clinically available antibody Eculizumab for C5);
reduction in the amount of inhibitor required to obtain desired inhibition level compared to C5-targeted therapy;
reduction of the risk of “breakthrough” as a consequence of increased synthesis of the target in the acute phase compared to C5-targeted therapy;
reduced side effects (e.g. blocking at the C7 stage allows production of C5a, an attractor and activator of phagocytic cells and is thus likely to confer lower risk of iatrogenic infection in treated individuals and hence be more suited to therapy of common, chronic diseases).

Embodiments of the invention will be further described in the detailed description. The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE FIGURES

The invention may be described with reference to the following figures in which:

FIG. 1 shows the results of a direct ELISA assay for binding of the monoclonal antibody 17E7 to C7 immobilised on the plastic well;

FIG. 2 shows the results of a standard classical pathway haemolysis assay using 17E7, SKY59 and Eculizumab in a series of dilutions to inhibit human serum-induced haemolysis of antibody-sensitised sheep erythrocytes (EA);

FIG. 3 shows the results of a serum excess classical pathway haemolysis assay using 17E7 and Eculizumab to inhibit human serum-induced haemolysis of EA when the serum concentration was increased closer to physiological;

FIG. 4 shows the results of a reactive lysis assay using 17E7 or 2H2 to inhibit haemolysis of guinea pig erythrocytes incubated sequentially with purified C5b6, C7, C8, and C9;

FIG. 5 shows the results of a western blot using 17E7 to detect C7 in human serum;

FIG. 6 shows the results of an in vivo complement inhibition assay using C7-deficient mice in which human C7 has been added to restore haemolytic activity (chequered bars 1 h after C7 administration), then inhibited by in vivo administration of 17E7 (horizontal striped bars, 17E7 (test) or control, mAb (control) given 1 h after C7 administration then bled after a further 2 h;

FIG. 7 shows the results of a direct ELISA assay for binding of 17E7 to non-human primate (cynomolgus) C7 immobilised on the plastic wells;

FIG. 8 shows the results of a direct ELISA assay for binding of 2H2 to C7;

FIG. 9 shows the results of a sandwich ELISA assay for binding of 2H2 to C7;

FIG. 10 shows the results of a standard classical pathway haemolysis assay using 2H2 and 7D4;

FIG. 11 shows the results of a western blot using 2H2 to detect human and rat C7;

FIG. 12 shows the results of a standard classical pathway haemolysis assay using various antibodies in a series of dilutions to inhibit human, mouse, rat or rabbit serum-induced haemolysis of antibody-sensitised sheep erythrocytes (EA);

FIG. 13 shows the results of a serum excess classical pathway haemolysis assay using 2H2 and 7D4 to inhibit rat serum-induced haemolysis of EA;

FIG. 14 shows the results of a reactive lysis assay using 2H2, 17E7, 59E7 or 73D1 to inhibit haemolysis of guinea pig erythrocytes incubated sequentially with purified C5b6, C7, C8, and C9;

FIG. 15 shows the results of a reactive lysis assay using control antibodies;

FIG. 16 shows the results of a direct ELISA assay for binding of the various antibodies to human C7 immobilised on the plastic well;

FIG. 17 shows the results of a direct ELISA assay for binding of the various antibodies to rat C7 immobilised on the plastic well;

FIG. 18 shows the results of a direct ELISA assay for binding of the various antibodies to mouse C7 immobilised on the plastic well;

FIG. 19 shows the results of a sandwich ELISA assay for binding of 2H2 to rat and human C7;

FIG. 20 shows the results of a competitive sandwich ELISA assay for binding of various antibodies to human C7 after binding of the 2H2 antibody (immobilised to plastic well as the capture) to C7;

FIG. 21 shows the results of a competitive sandwich ELISA assay for binding of various antibodies to human C7 after binding of the 17E7 antibody (immobilised to plastic well as the capture) to C7;

FIG. 22 shows the results of a competitive sandwich ELISA assay for binding of various antibodies to human C7 after binding of the 59E7 antibody (immobilised to plastic well as the capture) to C7;

FIG. 23 shows the results of a competitive sandwich ELISA assay for binding of various antibodies to human C7 after binding of the 73D1 antibody (immobilised to plastic well as the capture) to C7;

FIG. 24 shows the results of a competitive sandwich ELISA assay for binding of various antibodies to human C7 after binding of the polyclonal goat anti-C7 (Poly) antibody (immobilised to plastic well as the capture) to C7;

FIG. 25 shows the results of a western blot assay using various antibodies to detect human (Hu), monkey (Mk), mouse (Ms) and rat C7 under reducing (R) or non-reducing (NR) conditions;

FIG. 26 shows the results of a western blot pull-down assay using activated rat serum and 2H2;

FIG. 27 shows the results of a western blot pull-down assay using activated human serum and 2H2;

FIG. 28 shows the results of a sandwich ELISA assay for binding of 2H2 to various complement proteins in activated rat serum;

FIG. 29 shows the results of a sandwich ELISA assay for binding of 2H2, 17E7, 59 E7 and 73D1 to various complement proteins in activated human serum;

FIG. 30 shows the results of an in vivo complement inhibition assay;

FIG. 31 shows the results of a haemolysis assay using different doses of 2H2;

FIG. 32 shows weight loss of untreated rats and rats treated with 2H2 in a Myasthenia model;

FIG. 33 shows the clinical score of untreated rats and rats treated with 2H2 in a Myasthenia model;

FIG. 34 shows the results of a haemolytic assay at various time points in untreated rats and rats treated with 2H2 in a Myasthenia model;

FIG. 35 shows the mean number of neuromuscular junction endplates, stained with α-bungarotoxin (α-BuTx) to identify acetylcholine receptors (AchR), per field in untreated rats and rats treated with 2H2 in a Myasthenia model;

FIG. 36 shows α-BuTX mean intensity in untreated rats and rats treated with 2H2 in a Myasthenia model;

FIG. 37 shows C3b/iC3b mean intensity in untreated rats and rats treated with 2H2 in a Myasthenia model;

FIG. 38 shows C9/MAC mean intensity in untreated rats and rats treated with 2H2 in a Myasthenia model;

FIG. 39 is the ECACC certificate for the deposit of the hybridoma producing the 59E7 antibody (deposit no. 19080602) on 6 Aug. 2019;

FIG. 40 is the ECACC certificate for the deposit of the hybridoma producing the 73D1 antibody (deposit no. 19080601) on 6 Aug. 2019;

FIG. 41 is the ECACC certificate for the deposit of the hybridoma producing the 2H2 antibody (deposit no. 19080603) on 6 Aug. 2019;

FIG. 42 is the ECACC certificate for the deposit of the hybridoma producing the 17E7 antibody (deposit no. 19041601) on 16 Apr. 2019.

### DETAILED DESCRIPTION

There is provided herein an antibody or antibody fragment that binds to complement protein C7 in isolation or as part of a protein complex and inhibits complement activation.

The term “antibody” used herein refers to an immunoglobulin protein having two “heavy chain” subunits and two smaller “light chain” subunits. The four subunits are assembled to form two Fab (fragment antigen-binding) regions that each recognize and bind to a particular antigen (e.g. complement protein C7) and one Fc (fragment, crystallisable) region. Each Fab region is made from one heavy chain and one light chain, which each contributes one constant domain and one variable domain. Each Fc region is made from two heavy chains, which each contribute two or three constant domains.

The antibodies described herein may be any class of antibody, for example IgA, IgD, IgE, IgG, or IgM antibodies. In particular, the antibodies described herein may be IgD, IgE, or IgG antibodies. For example, the antibodies described herein may be IgG or IgM antibodies. For example, the antibodies described herein may be IgG antibodies.

The term “antibody fragment” used herein refers to a protein that comprises only part of the structure of an antibody but includes at least one region (a complementarity determining region) that recognizes and binds to a particular antigen (e.g. complement protein C7).

The antibody fragment may, for example, comprise, consist essentially of, or consist of one or more Fab region(s) (each made from one heavy chain and one light chain, which each contributes one constant domain and one variable domain). For example, the antibody fragment may comprise, consist essentially of, or consist of one Fab region. A single Fab region may be referred to as an antigen binding fragment (Fab). For example, the antibody fragment may comprise, consist essentially of, or consist of two Fab regions (F(ab)₂).

The antibody fragment may, for example, comprise, consist essentially of, or consist of one or more light chain variable regions and one or more heavy chain variable regions. The light chain variable regions and heavy chain variable regions may, for example, be part of the same polypeptide and may, for example, be joined by one or more flexible linker sequence(s). The antibody fragment may, for example, comprise, consist essentially of, or consist of one light chain variable region and one heavy chain variable region which may, for example, be part of the same polypeptide and may, for example, be joined by one or more flexible linker sequence(s). An antibody fragment comprising, consisting essentially of, or consisting of a light chain variable region and a heavy chain variable region on a single polypeptide may, for example, be referred to as a single-chain variable fragment (scFv). The scFvs may, for example, be engineered to non-covalently bind to other scFvs (which may be the same or different) to form bivalent molecules referred to as diabodies. The scFvs may, for example, comprise more than one light chain variable regions and/or more than one heavy chain variable regions on the same polypeptide (e.g. more than one scFv on the same polypeptide) and be referred to as tandem scFvs.

The antibody fragment may, for example, comprise, consist essentially of, or consist of a single heavy chain (including constant and variable domains) and a single light chain (including constant and variable domains). The constant domains of the heavy chain and the light chain may each independently be complete or truncated.

The antibody fragment may, for example, comprise, consist essentially of, or consist of two heavy chains (both including constant and variable domains) and two light chains (both including constant and variable domains) where the constant domains of one or more of the heavy and light chains are truncated.

The antibody fragment may, for example, comprise, consist essentially of, or consist of a single monomeric variable domain. These antibody fragments may, for example, be referred to as nanobodies. The antibody fragment may, for example, comprise, consist essentially of, or consist of a single heavy chain including one or more constant domains and one variable domain. The antibody fragment may, for example, comprise, consist essentially of, or consist of a single heavy chain including two constant domains and one variable domain.

The antibodies and antibody fragments of the present invention bind to complement protein C7 (by “bind” it is meant that the antibodies and antibody fragments are associated with complement protein C7 via non-covalent interactions such as electrostatic interactions, hydrogen bonds, Van der Waals forces, and hydrophobic interactions). The antibodies and antibody fragments may, for example, bind to complement protein C7 when it is part of a protein complex also containing other complement proteins (e.g. the complex C5b67, the complex C5b678, and/or the complex C5b67879n). Alternatively or additionally, the antibodies and antibody fragments may, for example, bind to complement protein C7 in isolation (i.e. when not in a complex with other complement proteins). In particular, the antibodies and antibody fragments may bind to C7 in the complex C5b67.

Complement protein C7 is a protein that is part of the complement system. There are a number of pathways for activation of complement that converge at a step whereby protein C5 is cleaved to release a fragment C5b. The protein fragment C5b binds to complement protein C6 to form the complex C5b6. The complex C5b6 then binds to complement protein C7 to form the complex C5b67. The complex C5b67 then associates with target cell membranes and binds complement protein C8 to insert the complex C5b678 further into the cell membrane. Finally, the complex C5b678 binds up to 18 copies of complement protein C9 which associate into a ring polymer inserted into the cell membrane to form a pore. The mature complex C5b6789n is referred to as the membrane attack complex (MAC).

Complement protein C7 is part of the “MAC family” of proteins. Complement protein C7 includes a membrane attack complex perforin domain and several tandemly arranged, cysteine-rich modules of less than 80 amino acid residues (including thrombospondin type, low density lipoprotein receptor class A, and modules similar in sequence to epidermal growth factor). Complement protein C7 also includes two approximately 60 residue complement control protein modules, followed by two cysteine-rich factor I-like modules composed of approximately 75 residues each. A description of the amino acid sequence and structure of human complement protein C7 can be found in Strausberg et al., “Complement component 7 [Homo sapiens]”, Proc. Natl. Acad. Sci. U.S.A., 99 (26), 16899-16903 (2002), or at https://www.ncbi.nlm.nih.gov/protein/AAH63851.1, the contents of which are incorporated herein by reference.

The antibodies and antibody fragments of the present invention may bind to human complement protein C7. Human complement protein C7 has the following amino acid sequence (SEQ ID NO: 9):

| | |
|---|---|
| 1 | mkvislfilv gfigefqsfs sasspvncqw dfyapwsecn gctktqtrrr svavygqyg |
| 61 | qpcvgnafet qsceptrgcp teegcgerfr cfsgqcisks lvcngdsdcd edsadedrce |
| 121 | dserrpscdi dkpppnielt gngyneltgq frnrvintks fggqcrkvfs gdgkdfyrls |
| 181 | gnvlsytfqv kinndfnyef ynstwsyvkh tstehtsssr krsffrssss ssrsytshtn |
| 241 | eihkgksyql lvventveva qfinnnpefl qlaepfwkel shlpslydys ayrrlidqyg |
| 301 | thylqsgslg geyrvlfyvd seklkqndfn sveekkckss gwhfvvkfss hgckelenal |
| 361 | kaasgtqnnv lrgepfirgg gagfisglsy leldnpagnk rrysawaesv tnlpqvikqk |
| 421 | ltplyelvke vpcasvkkly lkwaleeyld efdpchcrpc qngglatveg thclchckpy |
| 481 | tfgaaceqgv lvgnqaggvd ggwscwssws pcvqgkktrs recnnpppsg ggrscvgett |
| 541 | estqcedeel ehlrllephc fplslvptef cpsppalkdg fvqdegtmfp vgknvvytcn |
| 601 | egyslignpv arcgedlrwl vgemhcqkia cvlpvlmdgi qshpqkpfyt vgekvtvscs |
| 661 | ggmslegpsa flcgsslkws pemknarcvq kenpltqavp kcqrweklqn srcvckmpye |
| 721 | cgpsldvcaq derskrilpl tvckmhvlhc qgrnytltgr dsctlpasae kacgacplwg |
| 781 | kcdaesskcv creaseceee gfsicvevng keqtmsecea galrcrgqsi svtsirpcaa |
| 841 | etq |

Thus, in certain embodiments, the antibodies or antibody fragments of the present invention bind to SEQ ID NO: 9 or an amino acid sequence that is at least about 80% homologous to SEQ ID NO: 9. For example, the antibodies or antibody fragments may bind to an amino acid sequence that is at least about 82% or at least about 84% or at least about 85% or at least about 86% or at least about 88% or at least about 90% or at least about 92% or at least about 94% or at least about 95% or at least about 96 or at least about 98% or at least about 99% homologous to SEQ ID NO: 9. For example, the antibodies or antibody fragments may bind to an amino acid sequence that is up to 100% homologous to SEQ ID NO: 9.

In particular, the antibodies or antibody fragments of the present invention may bind to an epitope of SEQ ID NO: 9 or an epitope with an amino acid sequence that is at least about 90% homologous to the corresponding epitope of SEQ ID NO: 9, for example an epitope that is at least about 91% or at least about 92% or at least about 93% or at least about 94% or at least about 95% or at least about 96% or at least about 97% or at least about 98% or at least about 99% homologous to the corresponding epitope of SEQ ID NO: 9. For example, the antibodies or antibody fragments may bind to an epitope that is up to 100% homologous to the corresponding epitope of SEQ ID NO: 9.

Homology of amino acid sequences may be determined using the BLAST® (Basic Local Alignment Search Tool) provided by National Center for Biotechnology Information (NCBI).

The antibodies and antibody fragments of the present invention may bind to any particular region (epitope) of the complement C7 protein. In certain embodiments, the antibodies and antibody fragments of the present invention may bind to any epitope of the complement C7 protein involved in binding to a target cell membrane or involved in binding to other complement proteins in order to form the membrane attack complex (MAC). Thus, the antibodies and antibody fragments of the present invention may bind to any epitope of the complement C7 protein which results in inhibition of the formation of the membrane attack complex (MAC).

The antibodies and antibody fragments of the present invention may bind to any particular region (epitope) of the C7 protein as part of the C5b67 complex. This may, for example, prevent its insertion into the membrane. In certain embodiments, the antibodies and antibody fragments of the present invention may bind to any epitope of the C7 protein as part of the C5b67 complex involved in binding to a target cell membrane or involved in binding to other complement proteins in order to form the membrane attack complex (MAC). Thus, the antibodies and antibody fragments of the present invention may bind to any epitope of the C7 protein as part of the C5b67 complex which results in inhibition of the formation of the membrane attack complex (MAC).

In certain embodiments, the antibodies and antibody fragments of the present invention bind to one or both of the factor I-like modules (FIMs) at the C-terminus of the complement C7 protein.

The factor I-like modules (FIMs) of human complement protein C7 have the following amino acid sequence (SEQ ID NO: 10):

pltqavp kcqrweklqn srcvckmpye cgpsldvcaq derskrilpl tvckmhvlhc qgrnytltgr dsctlpasae kacgacplwg kcdaesskcv creaseceee gfsicvevng keqtmsecea galrcrgqsi svtsirpcaa etq

Thus, in certain embodiments, the antibodies or antibody fragments of the present invention bind to SEQ ID NO: 10 or an amino acid sequence that is at least about 80 homologous to SEQ ID NO: 10. For example, the antibodies or antibody fragments may bind to an amino acid sequence that is at least about 82% or at least about 84% or at least about 85% or at least about 86% or at least about 88% or at least about 90% or at least about 92% or at least about 94% or at least about 95% or at least about 96 or at least about 98% or at least about 99% homologous to SEQ ID NO: 10. For example, the antibodies or antibody fragments may bind to an amino acid sequence that is up to 100% homologous to SEQ ID NO: 10.

The antibodies and antibody fragments of the present invention bind C7 in isolation or as part of a protein complex and inhibit complement activation (i.e. inhibit activation of the complement system part of the immune system). For example, the antibodies and antibody fragments of the present invention may inhibit complement activation in vivo (i.e. in a subject such as a human subject). Inhibition of complement activation includes a reduction in complement activation and does not necessarily require complete inhibition, although in certain embodiments complete inhibition of complement activation may be obtained.

Inhibition of complement activation may be determined by performing a haemolysis assay using normal human serum (NHS) dosed with an antibody or antibody fragment of the present invention in vitro (e.g. as described in the examples below) using a molar ratio of antibody to C7 of 1:1 or less. Inhibition of complement activation is indicated by a reduction in serum lytic activity compared to serum lytic activity in the absence of the antibody or antibody fragment of the present invention.

For example, serum lytic activity may be reduced to levels equal to or less than about 25% of serum lytic activity in the absence of antibody or antibody fragment. For example, serum lytic activity may be reduced to levels equal to or less than about 20% or equal to or less than about 15% or equal to or less than about 10% of serum lytic activity in the absence of antibody or antibody fragment. For example, serum lytic activity may be reduced to levels equal to or greater than about 0.5% of serum lytic activity in the absence of antibody or antibody fragment or equal to or greater than about 1% of serum lytic activity in the absence of antibody or antibody fragment. Reduction of serum lytic activity to these levels may be indicative of an antibody or antibody fragment able to inhibit complement activity in vivo. Reduction of serum lytic activity to these levels may be indicative of an antibody or antibody fragment useful in a therapeutic method for treating or preventing a disease associated with dysregulation of complement.

Inhibition of complement activity may also be measured by measuring the level of production of terminal complexes such as measuring the level of production of the MAC complex. The antibodies and antibody fragments of the present invention may inhibit the formation of the membrane attack complex (MAC). Thus, inhibition of complement activity may be indicated by a decrease in the level of production of the MAC.

The antibodies and antibody fragments of the present invention may prevent binding of the complement protein C7 to the complement protein complex C5b6. This may be measured by performing a haemolysis assay as described in the examples below using the reactive lysis protocol wherein purified C5b6, C7, C8 and C9 are added in sequence to the target. Efficient inhibition of lysis when the antibody or antibody fragment is added before, but not after, C7 is added to target-associated C5b6 indicates that the antibody or antibody fragment prevents binding of complement protein C7 to complement protein complex C5b6.

Alternatively or additionally, the antibodies and antibody fragments of the present invention may prevent binding of the complement protein C8 to the C5b67 complex. Alternatively or additionally, the antibodies and antibody fragments of the present invention may prevent binding of the complex C5b67 to the target membrane. This may be measured by performing a haemolysis assay as described in the examples below using the reactive lysis protocol wherein purified C5b6, C7, C8, and C9 are added in sequence to the target. Efficient inhibition of lysis when the antibody or antibody fragment is added after C7 is added to target-associated C5b6 but before addition of C8 and C9 indicates that the antibody or antibody fragment prevents binding of complement protein to C8 to complement protein complex C5b67.

The antibodies and antibody fragments of the present invention may have a binding affinity constant (KD) for C7 in isolation or as part of a protein complex equal to or less than about 5×10⁻⁷ M. For example, the antibodies and antibody fragments may have a binding affinity constant (KD) for C7 in isolation or as part of a protein complex equal to or less than about 1×10⁻⁷ M or equal to or less than about 5×10⁻⁸ M or equal to or less than about 1×10⁻⁸ M or equal to or less than about 5×10⁻⁹ M or equal to or less than about 1×10⁻⁹ M. The antibodies and antibody fragments may, for example, have a binding affinity constant (KD) for C7 in isolation or as part of a protein complex equal to or greater than about 1×10⁻¹² M, for example equal to or greater than about 5×10⁻¹² M or equal to or greater than about 1×10⁻¹¹ M or equal to or greater than about 5×10⁻¹¹ M or equal to or greater than about 1×10⁻¹⁰ M or equal to or greater than about 5×10⁻¹⁰ M. For example, the antibodies and antibody fragments may have a binding affinity constant (KD) for C7 in isolation or as part of a protein complex ranging from about 1×10⁻¹² M to about 5×10⁻⁷ M or from about 1×10⁻¹¹ M to about 1×10⁻⁷ M or from about 1×10⁻¹⁹ M to about 5×10⁻⁸ M or from about 5×10⁻¹⁹ M to about 1×10⁻⁸ M or from about 5×10⁻¹⁰ M to about 5×10⁻⁹ M or from about 5×10⁻¹⁰ M to about 1×10⁻⁹ M.

The antibodies and antibody fragments of the present invention may have a binding association rate (ka) for C7 in isolation or as part of a protein complex equal to or greater than about 2.5×10³ M⁻¹ s⁻¹. For example, the antibodies and antibody fragments may have a binding association rate (ka) for C7 in isolation or as part of a protein complex equal to or greater than about 5×10³ M⁻¹ s⁻¹ or equal to or greater than about 1×10⁴ M⁻¹ s⁻¹ or equal to or greater than about 2×10⁴ M⁻¹ s⁻¹ or equal to or greater than about 3×10⁴ M⁻¹ s⁻¹ or equal to or greater than about 4×10⁴ M⁻¹ s⁻¹ or equal to or greater than about 5×10⁴ M⁻¹ s⁻¹ or equal to or greater than about 6×10⁴ M⁻¹ s⁻¹. For example, the antibodies and antibody fragments may have a binding association rate (ka) for C7 in isolation or as part of a protein complex equal to or less than about 1×10⁸ M⁻¹ s⁻¹ or equal to or less than about 1×10⁷ M⁻¹ s⁻¹ or equal to or less than about 1×10⁶ M⁻¹ s⁻¹ or equal to or less than about 1×10⁵ M⁻¹ s⁻¹. For example, the antibodies and antibody fragments may have a binding association rate (ka) for C7 ranging from about 2.5×10³ M⁻¹ s⁻¹ to about 1×10⁸ M⁻¹ s⁻¹ or from about 1×10⁴ M⁻¹ s⁻¹ to about 1×10⁷ M⁻¹ s⁻¹ or from about 3×10⁴ M⁻¹ s⁻¹ to about 1×10⁶ M⁻¹ s⁻¹ or from about 4×10⁴ M⁻¹ s⁻¹ to about 1×10⁵ M⁻¹ s⁻¹.

The antibodies and antibody fragments of the present invention may have a binding dissociation rate (kd) for C7 in isolation or as part of a protein complex equal to or less than about 5×10⁻³ s⁻¹. For example, the antibodies and antibody fragments may have a binding dissociation rate (kd) for C7 in isolation or as part of a protein complex equal to or less than about 1×10⁻³ s⁻¹ or equal to or less than about 5×10⁻⁴ s⁻¹ or equal to or less than about 1×10⁻⁴ s⁻¹ or equal to or less than about 9×10⁻⁵ s⁻¹ or equal to or less than about 8×10⁻⁵ s⁻¹ or equal to or less than about 7×10⁻⁵ s⁻¹. For example, the antibodies and antibody fragments may have a binding dissociation rate (kd) for C7 in isolation or as part of a protein complex equal to or greater than about 1×10⁻⁸ s⁻¹, for example equal to or greater than about 5×10⁻⁸ s⁻¹ or equal to or greater than about 1×10⁻⁷ s⁻¹ or equal to or greater than about 5×10⁻⁷ s⁻¹ or equal to or greater than about 1×10⁻⁶ s⁻¹ or equal to or greater than about 5×10⁻⁶ s⁻¹ or equal to or greater than about 1×10⁻⁵ s⁻¹. For example, the antibodies and antibody fragments may have a binding dissociation rate (kd) for C7 ranging from about 1×10⁻⁸ s⁻¹ to about 5×10⁻³ s⁻¹ or from about 1×10⁻⁸ s⁻¹ to about 5×10⁻⁴ s⁻¹ or from about 1×10⁻⁸ s⁻¹ to about 1×10⁻⁴ s⁻¹ or from about 1×10⁻⁷ s⁻¹ to about 9×10⁻⁵ s⁻¹ or from about 1×10⁻⁶ s⁻¹ to about 8×10⁻⁵ s⁻¹ or from about 1×10⁻⁵ s⁻¹ to about 7×10⁻⁵ s⁻¹.

Binding affinity constant (KD), binding association rate (ka) and binding dissociation rate (kd) may be measured by surface plasmon resonance using a Biacore™ assay system with antibody bound to the surface and C7 protein flowed over the surface or with C7 protein bound to the surface and antibody flowed over the surface, for example as described in the examples below. The antibody may, for example, be captured through Protein L (IgM antibodies) or Protein G (other antibodies) on a capture surface. The antibody may, for example, be captured through Protein G on a capture surface.

The antibodies and antibody fragments may comprise one or more amino acid sequences selected from the group consisting of amino acid sequences having at least about 60% homology to SEQ ID NO: 2, amino acid sequences having at least about 60% homology to SEQ ID NO: 3, and amino acid sequences having at least about 60% homology to SEQ ID NO: 4. These amino acid sequences may be referred to as complementarity determining regions. For example, the antibodies and antibody fragments may comprise two or more amino acid sequences selected from the group consisting of amino acid sequences having at least about 60% homology to SEQ ID NO: 2, amino acid sequences having at least about 60% homology to SEQ ID NO: 3, and amino acid sequences having at least about 60% homology to SEQ ID NO: 4. For example, the antibodies and antibody fragments may comprise three or more amino acid sequences selected from the group consisting of amino acid sequences having at least about 60% homology to SEQ ID NO: 2, amino acid sequences having at least about 60% homology to SEQ ID NO: 3, and amino acid sequences having at least about 60% homology to SEQ ID NO: 4.

The antibodies and antibody fragments may comprise one or more amino acid sequences selected from the group consisting of amino acid sequences having up to 3 amino acid differences to SEQ ID NO: 2, amino acid sequences having up to 3 amino acid differences to SEQ ID NO: 3, and amino acid sequences having up to 3 amino acid differences to SEQ ID NO: 4. These amino acid sequences may be referred to as complementarity determining regions. For example, the antibodies and antibody fragments may comprise two or more amino acid sequences selected from the group consisting of amino acid sequences having up to 3 amino acid differences to SEQ ID NO: 2, amino acid sequences having up to 3 amino acid differences to SEQ ID NO: 3, and amino acid sequences having up to 3 amino acid differences to SEQ ID NO: 4. For example, the antibodies and antibody fragments may comprise three or more amino acid sequences selected from the group consisting of amino acid sequences having up to 3 amino acid differences to SEQ ID NO: 2, amino acid sequences having up to 3 amino acid differences to SEQ ID NO: 3, and amino acid sequences having up to 3 amino acid differences to SEQ ID NO: 4.

For example, the antibodies and antibody fragments may comprise:
an amino acid sequence having at least about 60% homology to SEQ ID NO: 2; and/or
an amino acid sequence having at least about 60% homology to SEQ ID NO: 3; and/or
an amino acid sequence having at least about 60% homology to SEQ ID NO:4.

For example, the antibodies and antibody fragments may comprise:
an amino acid sequence having up to 3 amino acid differences to SEQ ID NO: 2; and/or
an amino acid sequence having up to 3 amino acid differences to SEQ ID NO: 3; and/or
an amino acid sequence having up to 3 amino acid differences to SEQ ID NO:4.

For example, the antibodies and antibody fragments may comprise:
an amino acid sequence having at least about 60% homology to SEQ ID NO: 2; and
an amino acid sequence having at least about 60% homology to SEQ ID NO: 3; and
an amino acid sequence having at least about 60% homology to SEQ ID NO:4.

For example, the antibodies and antibody fragments may comprise:
an amino acid sequence having up to 3 amino acid differences to SEQ ID NO: 2; and
an amino acid sequence having up to 3 amino acid differences to SEQ ID NO: 3; and
an amino acid sequence having up to 3 amino acid differences to SEQ ID NO:4.

For example, the antibodies and antibody fragments may comprise an amino acid sequence of SEQ ID NO: 2, an amino acid sequence of SEQ ID NO: 3, and an amino acid sequence of SEQ ID NO: 4.

SEQ ID NO: 2 or the amino acid sequence having at least 60% homology thereto or the amino acid sequence having up to 3 amino acid differences thereto may, for example, correspond to complementarity determining region (CDR) 1, for example on the heavy or light chain. SEQ ID NO: 3 or the amino acid sequence having at least 60% homology thereto or the amino acid sequence having up to 3 amino acid differences thereto may, for example, correspond to complementarity determining region (CDR) 2, for example on the heavy or light chain. SEQ ID NO: 4 or the amino acid sequence having at least 60% homology thereto or the amino acid sequence having up to 3 amino acid differences thereto may, for example, correspond to complementarity determining region (CDR) 3, for example on the heavy or light chain. SEQ ID NO: 2, SEQ ID NO: 3, and SEQ ID NO: 4 or the amino acid sequence having at least about 60% homology thereto or the amino acid sequence having up to 3 amino acid differences thereto may, for example, appear on the heavy chain of the antibody or antibody fragment.

The antibodies or antibody fragments may, for example, comprise two amino acid sequences having at least about 60% homology to SEQ ID NO: 2 and/or two amino acid sequences having at least about 60% homology to SEQ ID NO: 3 and/or two amino acid sequences having at least about 60% homology to SEQ ID NO: 4. For example, the antibodies or antibody fragments may comprise two amino acid sequences having at least about 60% homology to SEQ ID NO: 2 and two amino acid sequences having at least about 60% homology to SEQ ID NO: 3 and two amino acid sequences having at least about 60% homology to SEQ ID NO: 4. For example, the antibodies or antibody fragments may comprise two amino acid sequences of SEQ ID NO: 2 and/or two amino acid sequences of SEQ ID NO: 3 and/or two amino acid sequences of SEQ ID NO: 4. For example, the antibodies or antibody fragments may comprise two amino acid sequences of SEQ ID NO: 2 and two amino acid sequences of SEQ ID NO: 3 and two amino acid sequences of SEQ ID NO: 4.

The antibodies or antibody fragments may, for example, comprise two amino acid sequences having up to 3 amino acid differences to SEQ ID NO: 2 and/or two amino acid sequences having up to 3 amino acid differences to SEQ ID NO: 3 and/or two amino acid sequences having up to 3 amino acid differences to SEQ ID NO: 4. For example, the antibodies or antibody fragments may comprise two amino acid sequences having up to 3 amino acid differences to SEQ ID NO: 2 and two amino acid sequences having up to 3 amino acid differences to SEQ ID NO: 3 and two amino acid sequences having up to 3 amino acid differences to SEQ ID NO: 4. For example, the antibodies or antibody fragments may comprise two amino acid sequences of SEQ ID NO: 2 and/or two amino acid sequences of SEQ ID NO: 3 and/or two amino acid sequences of SEQ ID NO: 4. For example, the antibodies or antibody fragments may comprise two amino acid sequences of SEQ ID NO: 2 and two amino acid sequences of SEQ ID NO: 3 and two amino acid sequences of SEQ ID NO: 4.

In certain embodiments, the amino acid sequences having at least about 60% homology to SEQ ID NO: 2 or SEQ ID NO: 3 or SEQ ID NO: 4 each independently have a homology of at least about 65% or at least about 70% or at least about 75% or at least about 80% or at least about 85% or at least about 90% or at least about 95% or at least about 98% or at least about 99%. For example, the amino acid sequences having at least about 60% homology to SEQ ID NO: 2 or SEQ ID NO: 3 or SEQ ID NO: 4 may each independently have up a homology up to about 100%. In certain embodiments, the amino acid sequences having at least about 60% homology to SEQ ID NO: 2 or SEQ ID NO: 3, or SEQ ID NO: 4 each have a homology of at least about 80%.

In certain embodiments, the amino acid sequences having up to 3 amino acid differences to SEQ ID NO: 2 or SEQ ID NO: 3 or SEQ ID NO: 4 each independently have up to 2 amino acid differences or up to 1 amino acid differences to SEQ ID NO: 2 or SEQ ID NO: 3 or SEQ ID NO: 4. For example, the amino acid sequences having up to 3 amino acid differences to SEQ ID NO: 2 or SEQ ID NO: 3 or SEQ ID NO: 4 may each independently have 0 amino acid differences to SEQ ID NO: 2 or SEQ ID NO: 3 or SEQ ID NO: 4. In certain embodiments, the amino acid sequences having up to 3 amino acid differences to SEQ ID NO: 2 or SEQ ID NO: 3 or SEQ ID NO: 4 each have up to 2 amino acid differences or up to 1 amino acid difference to SEQ ID NO: 2, SEQ ID NO: 3, or SEQ ID NO: 4.

The antibodies and antibody fragments may comprise one or more amino acid sequences selected from the group consisting of amino acid sequences having at least about 60% homology to SEQ ID NO: 6, amino acid sequences having at least about 60% homology to SEQ ID NO: 7, and amino acid sequences having at least about 60% homology to SEQ ID NO: 8. These amino acid sequences may be referred to as complementarity determining regions. For example, the antibodies and antibody fragments may comprise two or more amino acid sequences selected from the group consisting of amino acid sequences having at least about 60% homology to SEQ ID NO: 6, amino acid sequences having at least about 60% homology to SEQ ID NO: 7, and amino acid sequences having at least about 60% homology to SEQ ID NO: 8. For example, the antibodies and antibody fragments may comprise three or more amino acid sequences selected from the group consisting of amino acid sequences having at least about 60% homology to SEQ ID NO: 6, amino acid sequences having at least about 60% homology to SEQ ID NO: 7, and amino acid sequences having at least about 60% homology to SEQ ID NO: 8.

The antibodies and antibody fragments may comprise one or more amino acid sequences selected from the group consisting of amino acid sequences having up to 3 amino acid differences to SEQ ID NO: 6, amino acid sequences having up to 3 amino acid differences to SEQ ID NO: 7, and amino acid sequences having up to 3 amino acid differences to SEQ ID NO: 8. These amino acid sequences may be referred to as complementarity determining regions. For example, the antibodies and antibody fragments may comprise two or more amino acid sequences selected from the group consisting of amino acid sequences having up to 3 amino acid differences to SEQ ID NO: 6, amino acid sequences having up to 3 amino acid differences to SEQ ID NO: 7, and amino acid sequences having up to 3 amino acid differences to SEQ ID NO: 8. For example, the antibodies and antibody fragments may comprise three or more amino acid sequences selected from the group consisting of amino acid sequences having up to 3 amino acid differences to SEQ ID NO: 6, amino acid sequences having up to 3 amino acid differences to SEQ ID NO: 7, and amino acid sequences having up to 3 amino acid differences to SEQ ID NO: 8.

For example, the antibodies and antibody fragments may comprise:
an amino acid sequence having at least about 60% homology to SEQ ID NO: 6; and/or
an amino acid sequence having at least about 60% homology to SEQ ID NO: 7; and/or
an amino acid sequence having at least about 60% homology to SEQ ID NO:8.

For example, the antibodies and antibody fragments may comprise:
an amino acid sequence having up to 3 amino acid differences to SEQ ID NO: 6; and/or
an amino acid sequence having up to 3 amino acid differences to SEQ ID NO: 7; and/or
an amino acid sequence having up to 3 amino acid differences to SEQ ID NO:8.

For example, the antibodies and antibody fragments may comprise:
an amino acid sequence having at least about 60% homology to SEQ ID NO: 6; and
an amino acid sequence having at least about 60% homology to SEQ ID NO: 7; and
an amino acid sequence having at least about 60% homology to SEQ ID NO: 8.

For example, the antibodies and antibody fragments may comprise:
an amino acid sequence having up to 3 amino acid differences to SEQ ID NO: 6; and
an amino acid sequence having up to 3 amino acid differences to SEQ ID NO: 7; and
an amino acid sequence having up to 3 amino acid differences to SEQ ID NO: 8.

For example, the antibodies and antibody fragments may comprise an amino acid sequence of SEQ ID NO: 6, an amino acid sequence of SEQ ID NO: 7, and an amino acid sequence of SEQ ID NO: 8.

SEQ ID NO: 6 or the amino acid sequence having at least 60% homology thereto or the amino acid sequence having up to 3 amino acid differences thereto may, for example, correspond to complementarity determining region (CDR) 1, for example on the heavy or light chain. SEQ ID NO: 7 or the amino acid sequence having at least 60% homology thereto or the amino acid sequence having up to 3 amino acid differences thereto may, for example, correspond to complementarity determining region (CDR) 2, for example on the heavy or light chain. SEQ ID NO: 8 or the amino acid sequence having at least 60% homology thereto or the amino acid sequence having up to 3 amino acid differences thereto may, for example, correspond to complementarity determining region (CDR) 3, for example on the heavy or light chain. SEQ ID NO: 6, SEQ ID NO: 7, and SEQ ID NO: 8 or the amino acid sequences having at least about 60% homology thereto or the amino acid sequence having up to 3 amino acid differences thereto may, for example, appear on the light chain of the antibody or antibody fragment.

The antibodies or antibody fragments may, for example, comprise two amino acid sequences having at least about 60% homology to SEQ ID NO: 6 and/or two amino acid sequences having at least about 60% homology to SEQ ID NO: 7 and/or two amino acid sequences having at least about 60% homology to SEQ ID NO: 8. For example, the antibodies or antibody fragments may comprise two amino acid sequences having at least about 60% homology to SEQ ID NO: 6 and two amino acid sequences having at least about 60% homology to SEQ ID NO: 7 and two amino acid sequences having at least about 60% homology to SEQ ID NO: 8. For example, the antibodies or antibody fragments may comprise two amino acid sequences of SEQ ID NO: 6 and/or two amino acid sequences of SEQ ID NO: 7 and/or two amino acid sequences of SEQ ID NO: 8. For example, the antibodies or antibody fragments may comprise two amino acid sequences of SEQ ID NO: 6 and two amino acid sequences of SEQ ID NO: 7 and two amino acid sequences of SEQ ID NO: 8.

The antibodies or antibody fragments may, for example, comprise two amino acid sequences having up to 3 amino acid differences to SEQ ID NO: 6 and/or two amino acid sequences having up to 3 amino acid differences to SEQ ID NO: 7 and/or two amino acid sequences having up to 3 amino acid differences to SEQ ID NO: 8. For example, the antibodies or antibody fragments may comprise two amino acid sequences having up to 3 amino acid differences to SEQ ID NO: 6 and two amino acid sequences having up to 3 amino acid differences to SEQ ID NO: 7 and two amino acid sequences having up to 3 amino acid differences to SEQ ID NO: 8. For example, the antibodies or antibody fragments may comprise two amino acid sequences of SEQ ID NO: 6 and/or two amino acid sequences of SEQ ID NO: 7 and/or two amino acid sequences of SEQ ID NO: 8. For example, the antibodies or antibody fragments may comprise two amino acid sequences of SEQ ID NO: 6 and two amino acid sequences of SEQ ID NO: 7 and two amino acid sequences of SEQ ID NO: 8.

In certain embodiments, the amino acid sequences having at least about 60% homology to SEQ ID NO: 6 or SEQ ID NO: 7 or SEQ ID NO: 8 each independently have a homology of at least about 65% or at least about 70% or at least about 75% or at least about 80% or at least about 85% or at least about 90% or at least about 95% or at least about 98% or at least about 99%. For example, the amino acid sequences having at least about 60% homology to SEQ ID NO: 6 or SEQ ID NO: 7 or SEQ ID NO: 8 may each independently have up a homology up to about 100%. In certain embodiments, the amino acid sequences having at least about 60% homology to SEQ ID NO: 6 or SEQ ID NO: 7, or SEQ ID NO: 8 each have a homology of at least about 80%.

In certain embodiments, the amino acid sequences having up to 3 amino acid differences to SEQ ID NO: 6 or SEQ ID NO: 7 or SEQ ID NO: 8 each independently have up to 2 amino acid differences or up to 1 amino acid differences to SEQ ID NO: 6 or SEQ ID NO: 7 or SEQ ID NO: 8. For example, the amino acid sequences having up to 3 amino acid differences to SEQ ID NO: 6 or SEQ ID NO: 7 or SEQ ID NO: 8 may each independently have 0 amino acid differences to SEQ ID NO: 6 or SEQ ID NO: 7 or SEQ ID NO: 8. In certain embodiments, the amino acid sequences having up to 3 amino acid differences to SEQ ID NO: 6 or SEQ ID NO: 7 or SEQ ID NO: 8 each have up to 2 amino acid differences or up to 1 amino acid difference to SEQ ID NO: 6, SEQ ID NO: 7, or SEQ ID NO: 8.

The antibody or antibody fragment may, for example, comprise an amino acid sequence having at least about 60% homology to SEQ ID NO: 1. For example, the antibody or antibody fragment may comprise an amino acid sequence having at least about 65% or at least about 70% or at least about 75% or at least about 80% or at least about 85% or at least about 90% or at least about 95% or at least about 98% or at least about 99% to SEQ ID NO: 1. For example, the antibody or antibody fragment may comprise an amino acid sequence having up to 100% homology to SEQ ID NO: 1. For example, the antibody or antibody fragment may comprise SEQ ID NO: 1. The antibody or antibody fragment may, for example, comprise two amino acid sequences having at least about 60% homology to SEQ ID NO: 1. For example, the antibody or antibody fragment may comprise two amino acid sequences of SEQ ID NO: 1.

SEQ ID NO: 1 or the sequence having at least about 60% homology thereto may, for example, occur on the heavy chain of the antibody or antibody fragment. SEQ ID NO: 1 may, for example, form part or all of a variable domain of the antibody or antibody fragment.

The antibody or antibody fragment may, for example, comprise an amino acid sequence having at least about 60% homology to SEQ ID NO: 5. For example, the antibody or antibody fragment may comprise an amino acid sequence having at least about 65% or at least about 70% or at least about 75% or at least about 80% or at least about 85% or at least about 90% or at least about 95% or at least about 98% or at least about 99% to SEQ ID NO: 5. For example, the antibody or antibody fragment may comprise an amino acid sequence having up to 100% homology to SEQ ID NO: 5. For example, the antibody or antibody fragment may comprise SEQ ID NO: 5. The antibody or antibody fragment may, for example, comprise two amino acid sequences having at least about 60% homology to SEQ ID NO: 5. For example, the antibody or antibody fragment may comprise two amino acid sequences of SEQ ID NO: 5.

SEQ ID NO: 5 or the sequence having at least about 60% homology thereto may, for example, occur on the light chain of the antibody or antibody fragment. SEQ ID NO: 5 may, for example, form part of a variable domain of the antibody or antibody fragment.

The antibodies and antibody fragments may comprise one or more amino acid sequences selected from the group consisting of amino acid sequences having at least about 60% homology to SEQ ID NO: 12, amino acid sequences having at least about 60% homology to SEQ ID NO: 13, and amino acid sequences having at least about 60% homology to SEQ ID NO: 14. These amino acid sequences may be referred to as complementarity determining regions. For example, the antibodies and antibody fragments may comprise two or more amino acid sequences selected from the group consisting of amino acid sequences having at least about 60% homology to SEQ ID NO: 12, amino acid sequences having at least about 60% homology to SEQ ID NO: 13, and amino acid sequences having at least about 60% homology to SEQ ID NO: 14. For example, the antibodies and antibody fragments may comprise three or more amino acid sequences selected from the group consisting of amino acid sequences having at least about 60% homology to SEQ ID NO: 12, amino acid sequences having at least about 60% homology to SEQ ID NO: 13, and amino acid sequences having at least about 60% homology to SEQ ID NO: 14.

The antibodies and antibody fragments may comprise one or more amino acid sequences selected from the group consisting of amino acid sequences having up to 3 amino acid differences to SEQ ID NO: 12, amino acid sequences having up to 3 amino acid differences to SEQ ID NO: 13, and amino acid sequences having up to 3 amino acid differences to SEQ ID NO: 14. These amino acid sequences may be referred to as complementarity determining regions. For example, the antibodies and antibody fragments may comprise two or more amino acid sequences selected from the group consisting of amino acid sequences having up to 3 amino acid differences to SEQ ID NO: 12, amino acid sequences having up to 3 amino acid differences to SEQ ID NO: 13, and amino acid sequences having up to 3 amino acid differences to SEQ ID NO: 14. For example, the antibodies and antibody fragments may comprise three or more amino acid sequences selected from the group consisting of amino acid sequences having up to 3 amino acid differences to SEQ ID NO: 12, amino acid sequences having up to 3 amino acid differences to SEQ ID NO: 13, and amino acid sequences having up to 3 amino acid differences to SEQ ID NO: 14.

For example, the antibodies and antibody fragments may comprise:
an amino acid sequence having at least about 60% homology to SEQ ID NO: 12; and/or
an amino acid sequence having at least about 60% homology to SEQ ID NO: 13; and/or
an amino acid sequence having at least about 60% homology to SEQ ID NO: 14.

For example, the antibodies and antibody fragments may comprise:
an amino acid sequence having up to 3 amino acid differences to SEQ ID NO: 12; and/or
an amino acid sequence having up to 3 amino acid differences to SEQ ID NO: 13; and/or
an amino acid sequence having up to 3 amino acid differences to SEQ ID NO: 14.

For example, the antibodies and antibody fragments may comprise:
an amino acid sequence having at least about 60% homology to SEQ ID NO: 12; and
an amino acid sequence having at least about 60% homology to SEQ ID NO: 13; and
an amino acid sequence having at least about 60% homology to SEQ ID NO: 14.

For example, the antibodies and antibody fragments may comprise:
an amino acid sequence having up to 3 amino acid differences to SEQ ID NO: 12; and
an amino acid sequence having up to 3 amino acid differences to SEQ ID NO: 13; and
an amino acid sequence having up to 3 amino acid differences to SEQ ID NO: 14.

For example, the antibodies and antibody fragments may comprise an amino acid sequence of SEQ ID NO: 12, an amino acid sequence of SEQ ID NO: 13, and an amino acid sequence of SEQ ID NO: 14.

SEQ ID NO: 12 or the amino acid sequence having at least 60% homology thereto or the amino acid sequence having up to 3 amino acid differences thereto may, for example, correspond to complementarity determining region (CDR) 1, for example on the heavy or light chain. SEQ ID NO: 13 or the amino acid sequence having at least 60% homology thereto or the amino acid sequence having up to 3 amino acid differences thereto may, for example, correspond to complementarity determining region (CDR) 2, for example on the heavy or light chain. SEQ ID NO: 14 or the amino acid sequence having at least 60% homology thereto or the amino acid sequence having up to 3 amino acid differences thereto may, for example, correspond to complementarity determining region (CDR) 3, for example on the heavy or light chain. SEQ ID NO: 12, SEQ ID NO:13, and SEQ ID NO: 14 or the amino acid sequence having at least about 60% homology thereto or the amino acid sequence having up to 3 amino acid differences thereto may, for example, appear on the heavy chain of the antibody or antibody fragment.

The antibodies or antibody fragments may, for example, comprise two amino acid sequences having at least about 60% homology to SEQ ID NO: 12 and/or two amino acid sequences having at least about 60% homology to SEQ ID NO: 13 and/or two amino acid sequences having at least about 60% homology to SEQ ID NO: 14. For example, the antibodies or antibody fragments may comprise two amino acid sequences having at least about 60% homology to SEQ ID NO: 12 and two amino acid sequences having at least about 60% homology to SEQ ID NO: 13 and two amino acid sequences having at least about 60% homology to SEQ ID NO: 14. For example, the antibodies or antibody fragments may comprise two amino acid sequences of SEQ ID NO: 12 and/or two amino acid sequences of SEQ ID NO: 13 and/or two amino acid sequences of SEQ ID NO: 14. For example, the antibodies or antibody fragments may comprise two amino acid sequences of SEQ ID NO: 12 and two amino acid sequences of SEQ ID NO: 13 and two amino acid sequences of SEQ ID NO: 14.

The antibodies or antibody fragments may, for example, comprise two amino acid sequences having up to 3 amino acid differences to SEQ ID NO: 12 and/or two amino acid sequences having up to 3 amino acid differences to SEQ ID NO: 13 and/or two amino acid sequences having up to 3 amino acid differences to SEQ ID NO: 14. For example, the antibodies or antibody fragments may comprise two amino acid sequences having up to 3 amino acid differences to SEQ ID NO: 12 and two amino acid sequences having up to 3 amino acid differences to SEQ ID NO: 13 and two amino acid sequences having up to 3 amino acid differences to SEQ ID NO: 14. For example, the antibodies or antibody fragments may comprise two amino acid sequences of SEQ ID NO: 12 and/or two amino acid sequences of SEQ ID NO: 13 and/or two amino acid sequences of SEQ ID NO: 14. For example, the antibodies or antibody fragments may comprise two amino acid sequences of SEQ ID NO: 12 and two amino acid sequences of SEQ ID NO: 13 and two amino acid sequences of SEQ ID NO: 14.

In certain embodiments, the amino acid sequences having at least about 60% homology to SEQ ID NO: 12 or SEQ ID NO: 13 or SEQ ID NO: 14 each independently have a homology of at least about 65% or at least about 70% or at least about 75% or at least about 80% or at least about 85% or at least about 90% or at least about 95% or at least about 98% or at least about 99%. For example, the amino acid sequences having at least about 60% homology to SEQ ID NO: 12 or SEQ ID NO: 13 or SEQ ID NO: 14 may each independently have up a homology up to about 100%. In certain embodiments, the amino acid sequences having at least about 60% homology to SEQ ID NO: 12 or SEQ ID NO: 13, or SEQ ID NO: 14 each have a homology of at least about 80%.

In certain embodiments, the amino acid sequences having up to 3 amino acid differences to SEQ ID NO: 12 or SEQ ID NO: 13 or SEQ ID NO: 14 each independently have up to 2 amino acid differences or up to 1 amino acid differences to SEQ ID NO: 12 or SEQ ID NO: 13 or SEQ ID NO: 14. For example, the amino acid sequences having up to 3 amino acid differences to SEQ ID NO: 12 or SEQ ID NO: 13 or SEQ ID NO: 14 may each independently have 0 amino acid differences to SEQ ID NO: 12 or SEQ ID NO: 13 or SEQ ID NO: 14. In certain embodiments, the amino acid sequences having up to 3 amino acid differences to SEQ ID NO: 12 or SEQ ID NO: 13 or SEQ ID NO: 14 each have up to 2 amino acid differences or up to 1 amino acid difference to SEQ ID NO: 12, SEQ ID NO: 13, or SEQ ID NO: 14.

The antibodies and antibody fragments may comprise one or more amino acid sequences selected from the group consisting of amino acid sequences having at least about 60% homology to SEQ ID NO: 16, amino acid sequences having at least about 60% homology to SEQ ID NO: 17, and amino acid sequences having at least about 60% homology to SEQ ID NO: 18. These amino acid sequences may be referred to as complementarity determining regions. For example, the antibodies and antibody fragments may comprise two or more amino acid sequences selected from the group consisting of amino acid sequences having at least about 60% homology to SEQ ID NO: 16, amino acid sequences having at least about 60% homology to SEQ ID NO: 17, and amino acid sequences having at least about 60% homology to SEQ ID NO: 18. For example, the antibodies and antibody fragments may comprise three or more amino acid sequences selected from the group consisting of amino acid sequences having at least about 60% homology to SEQ ID NO: 16, amino acid sequences having at least about 60% homology to SEQ ID NO: 17, and amino acid sequences having at least about 60% homology to SEQ ID NO: 18.

The antibodies and antibody fragments may comprise one or more amino acid sequences selected from the group consisting of amino acid sequences having up to 3 amino acid differences to SEQ ID NO: 16, amino acid sequences having up to 3 amino acid differences to SEQ ID NO: 17, and amino acid sequences having up to 3 amino acid differences to SEQ ID NO: 18. These amino acid sequences may be referred to as complementarity determining regions. For example, the antibodies and antibody fragments may comprise two or more amino acid sequences selected from the group consisting of amino acid sequences having up to 3 amino acid differences to SEQ ID NO: 16, amino acid sequences having up to 3 amino acid differences to SEQ ID NO: 17, and amino acid sequences having up to 3 amino acid differences to SEQ ID NO: 18. For example, the antibodies and antibody fragments may comprise three or more amino acid sequences selected from the group consisting of amino acid sequences having up to 3 amino acid differences to SEQ ID NO: 16, amino acid sequences having up to 3 amino acid differences to SEQ ID NO: 17, and amino acid sequences having up to 3 amino acid differences to SEQ ID NO: 18.

For example, the antibodies and antibody fragments may comprise:
an amino acid sequence having at least about 60% homology to SEQ ID NO: 16; and/or
an amino acid sequence having at least about 60% homology to SEQ ID NO: 17; and/or
an amino acid sequence having at least about 60% homology to SEQ ID NO: 18.

For example, the antibodies and antibody fragments may comprise:
an amino acid sequence having up to 3 amino acid differences to SEQ ID NO: 16; and/or
an amino acid sequence having up to 3 amino acid differences to SEQ ID NO: 17; and/or
an amino acid sequence having up to 3 amino acid differences to SEQ ID NO: 18.

For example, the antibodies and antibody fragments may comprise:
an amino acid sequence having at least about 60% homology to SEQ ID NO: 16; and
an amino acid sequence having at least about 60% homology to SEQ ID NO: 17; and
an amino acid sequence having at least about 60% homology to SEQ ID NO: 18.

For example, the antibodies and antibody fragments may comprise:
an amino acid sequence having up to 3 amino acid differences to SEQ ID NO: 16; and
an amino acid sequence having up to 3 amino acid differences to SEQ ID NO: 17; and
an amino acid sequence having up to 3 amino acid differences to SEQ ID NO: 18.

For example, the antibodies and antibody fragments may comprise an amino acid sequence of SEQ ID NO: 16, an amino acid sequence of SEQ ID NO: 17, and an amino acid sequence of SEQ ID NO: 18.

SEQ ID NO: 16 or the amino acid sequence having at least 60% homology thereto or the amino acid sequence having up to 3 amino acid differences thereto may, for example, correspond to complementarity determining region (CDR) 1, for example on the heavy or light chain. SEQ ID NO: 17 or the amino acid sequence having at least 60% homology thereto or the amino acid sequence having up to 3 amino acid differences thereto may, for example, correspond to complementarity determining region (CDR) 2, for example on the heavy or light chain. SEQ ID NO: 18 or the amino acid sequence having at least 60% homology thereto or the amino acid sequence having up to 3 amino acid differences thereto may, for example, correspond to complementarity determining region (CDR) 3, for example on the heavy or light chain. SEQ ID NO: 16, SEQ ID NO: 17, and SEQ ID NO: 18 or the amino acid sequences having at least about 60% homology thereto or the amino acid sequence having up to 3 amino acid differences thereto may, for example, appear on the light chain of the antibody or antibody fragment.

The antibodies or antibody fragments may, for example, comprise two amino acid sequences having at least about 60% homology to SEQ ID NO: 16 and/or two amino acid sequences having at least about 60% homology to SEQ ID NO: 17 and/or two amino acid sequences having at least about 60% homology to SEQ ID NO: 18. For example, the antibodies or antibody fragments may comprise two amino acid sequences having at least about 60% homology to SEQ ID NO: 16 and two amino acid sequences having at least about 60% homology to SEQ ID NO: 17 and two amino acid sequences having at least about 60% homology to SEQ ID NO: 18. For example, the antibodies or antibody fragments may comprise two amino acid sequences of SEQ ID NO: 16 and/or two amino acid sequences of SEQ ID NO: 17 and/or two amino acid sequences of SEQ ID NO: 18. For example, the antibodies or antibody fragments may comprise two amino acid sequences of SEQ ID NO: 16 and two amino acid sequences of SEQ ID NO: 17 and two amino acid sequences of SEQ ID NO: 18.

The antibodies or antibody fragments may, for example, comprise two amino acid sequences having up to 3 amino acid differences to SEQ ID NO: 16 and/or two amino acid sequences having up to 3 amino acid differences to SEQ ID NO: 17 and/or two amino acid sequences having up to 3 amino acid differences to SEQ ID NO: 18. For example, the antibodies or antibody fragments may comprise two amino acid sequences having up to 3 amino acid differences to SEQ ID NO: 16 and two amino acid sequences having up to 3 amino acid differences to SEQ ID NO: 17 and two amino acid sequences having up to 3 amino acid differences to SEQ ID NO: 18. For example, the antibodies or antibody fragments may comprise two amino acid sequences of SEQ ID NO: 16 and/or two amino acid sequences of SEQ ID NO: 17 and/or two amino acid sequences of SEQ ID NO: 18. For example, the antibodies or antibody fragments may comprise two amino acid sequences of SEQ ID NO: 16 and two amino acid sequences of SEQ ID NO: 17 and two amino acid sequences of SEQ ID NO: 18.

In certain embodiments, the amino acid sequences having at least about 60% homology to SEQ ID NO: 16 or SEQ ID NO: 17 or SEQ ID NO: 18 each independently have a homology of at least about 65% or at least about 70% or at least about 75% or at least about 80% or at least about 85% or at least about 90% or at least about 95% or at least about 98% or at least about 99%. For example, the amino acid sequences having at least about 60% homology to SEQ ID NO: 16 or SEQ ID NO: 17 or SEQ ID NO: 18 may each independently have up a homology up to about 100%. In certain embodiments, the amino acid sequences having at least about 60% homology to SEQ ID NO: 16 or SEQ ID NO: 17, or SEQ ID NO: 18 each have a homology of at least about 80%.

In certain embodiments, the amino acid sequences having up to 3 amino acid differences to SEQ ID NO: 16 or SEQ ID NO: 17 or SEQ ID NO: 18 each independently have up to 2 amino acid differences or up to 1 amino acid differences to SEQ ID NO: 16 or SEQ ID NO: 17 or SEQ ID NO: 18. For example, the amino acid sequences having up to 3 amino acid differences to SEQ ID NO: 16 or SEQ ID NO: 17 or SEQ ID NO: 18 may each independently have 0 amino acid differences to SEQ ID NO: 16 or SEQ ID NO: 17 or SEQ ID NO: 18. In certain embodiments, the amino acid sequences having up to 3 amino acid differences to SEQ ID NO: 16 or SEQ ID NO: 17 or SEQ ID NO: 18 each have up to 2 amino acid differences or up to 1 amino acid difference to SEQ ID NO: 16, SEQ ID NO: 17, or SEQ ID NO: 18.

The antibody or antibody fragment may, for example, comprise an amino acid sequence having at least about 60% homology to SEQ ID NO: 11. For example, the antibody or antibody fragment may comprise an amino acid sequence having at least about 65% or at least about 70% or at least about 75% or at least about 80% or at least about 85% or at least about 90% or at least about 95% or at least about 98% or at least about 99% to SEQ ID NO: 11. For example, the antibody or antibody fragment may comprise an amino acid sequence having up to 100% homology to SEQ ID NO: 11. For example, the antibody or antibody fragment may comprise SEQ ID NO: 11. The antibody or antibody fragment may, for example, comprise two amino acid sequences having at least about 60% homology to SEQ ID NO: 11. For example, the antibody or antibody fragment may comprise two amino acid sequences of SEQ ID NO: 11.

SEQ ID NO: 11 or the sequence having at least about 60% homology thereto may, for example, occur on the heavy chain of the antibody or antibody fragment. SEQ ID NO: 11 may, for example, form part or all of a variable domain of the antibody or antibody fragment.

The antibody or antibody fragment may, for example, comprise an amino acid sequence having at least about 60% homology to SEQ ID NO: 15. For example, the antibody or antibody fragment may comprise an amino acid sequence having at least about 65% or at least about 70% or at least about 75% or at least about 80% or at least about 85% or at least about 90% or at least about 95% or at least about 98% or at least about 99% to SEQ ID NO: 15. For example, the antibody or antibody fragment may comprise an amino acid sequence having up to 100% homology to SEQ ID NO: 15. For example, the antibody or antibody fragment may comprise SEQ ID NO: 15. The antibody or antibody fragment may, for example, comprise two amino acid sequences having at least about 60% homology to SEQ ID NO: 15. For example, the antibody or antibody fragment may comprise two amino acid sequences of SEQ ID NO: 15.

SEQ ID NO: 15 or the sequence having at least about 60% homology thereto may, for example, occur on the light chain of the antibody or antibody fragment. SEQ ID NO: 15 may, for example, form part of a variable domain of the antibody or antibody fragment.

The antibodies and antibody fragments may comprise one or more amino acid sequences selected from the group consisting of amino acid sequences having at least about 60% homology to SEQ ID NO: 20, amino acid sequences having at least about 60% homology to SEQ ID NO: 21, and amino acid sequences having at least about 60% homology to SEQ ID NO: 22. These amino acid sequences may be referred to as complementarity determining regions. For example, the antibodies and antibody fragments may comprise two or more amino acid sequences selected from the group consisting of amino acid sequences having at least about 60% homology to SEQ ID NO: 20, amino acid sequences having at least about 60% homology to SEQ ID NO: 21, and amino acid sequences having at least about 60% homology to SEQ ID NO: 22. For example, the antibodies and antibody fragments may comprise three or more amino acid sequences selected from the group consisting of amino acid sequences having at least about 60% homology to SEQ ID NO: 20, amino acid sequences having at least about 60% homology to SEQ ID NO: 21, and amino acid sequences having at least about 60% homology to SEQ ID NO: 22.

The antibodies and antibody fragments may comprise one or more amino acid sequences selected from the group consisting of amino acid sequences having up to 3 amino acid differences to SEQ ID NO: 20, amino acid sequences having up to 3 amino acid differences to SEQ ID NO: 21, and amino acid sequences having up to 3 amino acid differences to SEQ ID NO: 22. These amino acid sequences may be referred to as complementarity determining regions. For example, the antibodies and antibody fragments may comprise two or more amino acid sequences selected from the group consisting of amino acid sequences having up to 3 amino acid differences to SEQ ID NO: 20, amino acid sequences having up to 3 amino acid differences to SEQ ID NO: 21, and amino acid sequences having up to 3 amino acid differences to SEQ ID NO: 22. For example, the antibodies and antibody fragments may comprise three or more amino acid sequences selected from the group consisting of amino acid sequences having up to 3 amino acid differences to SEQ ID NO: 20, amino acid sequences having up to 3 amino acid differences to SEQ ID NO: 21, and amino acid sequences having up to 3 amino acid differences to SEQ ID NO: 22.

For example, the antibodies and antibody fragments may comprise:
an amino acid sequence having at least about 60% homology to SEQ ID NO: 20; and/or
an amino acid sequence having at least about 60% homology to SEQ ID NO: 21; and/or
an amino acid sequence having at least about 60% homology to SEQ ID NO:22.

For example, the antibodies and antibody fragments may comprise:
an amino acid sequence having up to 3 amino acid differences to SEQ ID NO: 20; and/or
an amino acid sequence having up to 3 amino acid differences to SEQ ID NO: 21; and/or
an amino acid sequence having up to 3 amino acid differences to SEQ ID NO:22.

For example, the antibodies and antibody fragments may comprise:
an amino acid sequence having at least about 60% homology to SEQ ID NO: 20; and
an amino acid sequence having at least about 60% homology to SEQ ID NO: 21; and
an amino acid sequence having at least about 60% homology to SEQ ID NO:22.

For example, the antibodies and antibody fragments may comprise:
an amino acid sequence having up to 3 amino acid differences to SEQ ID NO: 20; and
an amino acid sequence having up to 3 amino acid differences to SEQ ID NO: 21; and
an amino acid sequence having up to 3 amino acid differences to SEQ ID NO:22.

For example, the antibodies and antibody fragments may comprise an amino acid sequence of SEQ ID NO: 20, an amino acid sequence of SEQ ID NO: 21, and an amino acid sequence of SEQ ID NO: 22.

SEQ ID NO: 20 or the amino acid sequence having at least 60% homology thereto or the amino acid sequence having up to 3 amino acid differences thereto may, for example, correspond to complementarity determining region (CDR) 1, for example on the heavy or light chain. SEQ ID NO: 21 or the amino acid sequence having at least 60% homology thereto or the amino acid sequence having up to 3 amino acid differences thereto may, for example, correspond to complementarity determining region (CDR) 2, for example on the heavy or light chain. SEQ ID NO: 22 or the amino acid sequence having at least 60% homology thereto or the amino acid sequence having up to 3 amino acid differences thereto may, for example, correspond to complementarity determining region (CDR) 3, for example on the heavy or light chain. SEQ ID NO: 20, SEQ ID NO: 21, and SEQ ID NO: 22 or the amino acid sequence having at least about 60% homology thereto or the amino acid sequence having up to 3 amino acid differences thereto may, for example, appear on the heavy chain of the antibody or antibody fragment.

The antibodies or antibody fragments may, for example, comprise two amino acid sequences having at least about 60% homology to SEQ ID NO: 20 and/or two amino acid sequences having at least about 60% homology to SEQ ID NO: 21 and/or two amino acid sequences having at least about 60% homology to SEQ ID NO: 22. For example, the antibodies or antibody fragments may comprise two amino acid sequences having at least about 60% homology to SEQ ID NO: 20 and two amino acid sequences having at least about 60% homology to SEQ ID NO: 21 and two amino acid sequences having at least about 60% homology to SEQ ID NO: 22. For example, the antibodies or antibody fragments may comprise two amino acid sequences of SEQ ID NO: 20 and/or two amino acid sequences of SEQ ID NO: 21 and/or two amino acid sequences of SEQ ID NO: 22. For example, the antibodies or antibody fragments may comprise two amino acid sequences of SEQ ID NO: 20 and two amino acid sequences of SEQ ID NO: 21 and two amino acid sequences of SEQ ID NO: 22.

The antibodies or antibody fragments may, for example, comprise two amino acid sequences having up to 3 amino acid differences to SEQ ID NO: 20 and/or two amino acid sequences having up to 3 amino acid differences to SEQ ID NO: 21 and/or two amino acid sequences having up to 3 amino acid differences to SEQ ID NO: 22. For example, the antibodies or antibody fragments may comprise two amino acid sequences having up to 3 amino acid differences to SEQ ID NO: 20 and two amino acid sequences having up to 3 amino acid differences to SEQ ID NO: 21 and two amino acid sequences having up to 3 amino acid differences to SEQ ID NO: 22. For example, the antibodies or antibody fragments may comprise two amino acid sequences of SEQ ID NO: 20 and/or two amino acid sequences of SEQ ID NO: 21 and/or two amino acid sequences of SEQ ID NO: 22. For example, the antibodies or antibody fragments may comprise two amino acid sequences of SEQ ID NO: 20 and two amino acid sequences of SEQ ID NO: 21 and two amino acid sequences of SEQ ID NO: 22.

In certain embodiments, the amino acid sequences having at least about 60% homology to SEQ ID NO: 20 or SEQ ID NO: 21 or SEQ ID NO: 22 each independently have a homology of at least about 65% or at least about 70% or at least about 75% or at least about 80% or at least about 85% or at least about 90% or at least about 95% or at least about 98% or at least about 99%. For example, the amino acid sequences having at least about 60% homology to SEQ ID NO: 20 or SEQ ID NO: 21 or SEQ ID NO: 22 may each independently have up a homology up to about 100%. In certain embodiments, the amino acid sequences having at least about 60% homology to SEQ ID NO: 20 or SEQ ID NO: 21, or SEQ ID NO: 22 each have a homology of at least about 80%.

In certain embodiments, the amino acid sequences having up to 3 amino acid differences to SEQ ID NO: 20 or SEQ ID NO: 21 or SEQ ID NO: 22 each independently have up to 2 amino acid differences or up to 1 amino acid differences to SEQ ID NO: 20 or SEQ ID NO: 21 or SEQ ID NO: 22. For example, the amino acid sequences having up to 3 amino acid differences to SEQ ID NO: 20 or SEQ ID NO: 21 or SEQ ID NO: 22 may each independently have 0 amino acid differences to SEQ ID NO: 20 or SEQ ID NO: 21 or SEQ ID NO: 22. In certain embodiments, the amino acid sequences having up to 3 amino acid differences to SEQ ID NO: 20 or SEQ ID NO: 21 or SEQ ID NO: 22 each have up to 2 amino acid differences or up to 1 amino acid difference to SEQ ID NO: 20, SEQ ID NO: 21, or SEQ ID NO: 22.

The antibodies and antibody fragments may comprise one or more amino acid sequences selected from the group consisting of amino acid sequences having at least about 60% homology to SEQ ID NO: 24, amino acid sequences having at least about 60% homology to SEQ ID NO: 25, and amino acid sequences having at least about 60% homology to SEQ ID NO: 26. These amino acid sequences may be referred to as complementarity determining regions. For example, the antibodies and antibody fragments may comprise two or more amino acid sequences selected from the group consisting of amino acid sequences having at least about 60% homology to SEQ ID NO: 24, amino acid sequences having at least about 60% homology to SEQ ID NO: 25, and amino acid sequences having at least about 60% homology to SEQ ID NO: 26. For example, the antibodies and antibody fragments may comprise three or more amino acid sequences selected from the group consisting of amino acid sequences having at least about 60% homology to SEQ ID NO: 24, amino acid sequences having at least about 60% homology to SEQ ID NO: 25, and amino acid sequences having at least about 60% homology to SEQ ID NO: 26.

The antibodies and antibody fragments may comprise one or more amino acid sequences selected from the group consisting of amino acid sequences having up to 3 amino acid differences to SEQ ID NO: 24, amino acid sequences having up to 3 amino acid differences to SEQ ID NO: 25, and amino acid sequences having up to 3 amino acid differences to SEQ ID NO: 26. These amino acid sequences may be referred to as complementarity determining regions. For example, the antibodies and antibody fragments may comprise two or more amino acid sequences selected from the group consisting of amino acid sequences having up to 3 amino acid differences to SEQ ID NO: 24, amino acid sequences having up to 3 amino acid differences to SEQ ID NO: 25, and amino acid sequences having up to 3 amino acid differences to SEQ ID NO: 26. For example, the antibodies and antibody fragments may comprise three or more amino acid sequences selected from the group consisting of amino acid sequences having up to 3 amino acid differences to SEQ ID NO: 24, amino acid sequences having up to 3 amino acid differences to SEQ ID NO: 25, and amino acid sequences having up to 3 amino acid differences to SEQ ID NO: 26.

For example, the antibodies and antibody fragments may comprise:
an amino acid sequence having at least about 60% homology to SEQ ID NO: 24; and/or
an amino acid sequence having at least about 60% homology to SEQ ID NO: 25; and/or
an amino acid sequence having at least about 60% homology to SEQ ID NO:26.

For example, the antibodies and antibody fragments may comprise:
an amino acid sequence having up to 3 amino acid differences to SEQ ID NO: 24; and/or
an amino acid sequence having up to 3 amino acid differences to SEQ ID NO: 25; and/or
an amino acid sequence having up to 3 amino acid differences to SEQ ID NO: 26.

For example, the antibodies and antibody fragments may comprise:
an amino acid sequence having at least about 60% homology to SEQ ID NO: 24; and
an amino acid sequence having at least about 60% homology to SEQ ID NO: 25; and
an amino acid sequence having at least about 60% homology to SEQ ID NO: 26.

For example, the antibodies and antibody fragments may comprise:
an amino acid sequence having up to 3 amino acid differences to SEQ ID NO: 24; and
an amino acid sequence having up to 3 amino acid differences to SEQ ID NO: 25; and
an amino acid sequence having up to 3 amino acid differences to SEQ ID NO: 26.

For example, the antibodies and antibody fragments may comprise an amino acid sequence of SEQ ID NO: 24, an amino acid sequence of SEQ ID NO: 25, and an amino acid sequence of SEQ ID NO: 26.

SEQ ID NO: 24 or the amino acid sequence having at least 60% homology thereto or the amino acid sequence having up to 3 amino acid differences thereto may, for example, correspond to complementarity determining region (CDR) 1, for example on the heavy or light chain. SEQ ID NO: 25 or the amino acid sequence having at least 60% homology thereto or the amino acid sequence having up to 3 amino acid differences thereto may, for example, correspond to complementarity determining region (CDR) 2, for example on the heavy or light chain. SEQ ID NO: 26 or the amino acid sequence having at least 60% homology thereto or the amino acid sequence having up to 3 amino acid differences thereto may, for example, correspond to complementarity determining region (CDR) 3, for example on the heavy or light chain. SEQ ID NO: 24, SEQ ID NO: 25, and SEQ ID NO: 26 or the amino acid sequences having at least about 60% homology thereto or the amino acid sequence having up to 3 amino acid differences thereto may, for example, appear on the light chain of the antibody or antibody fragment.

The antibodies or antibody fragments may, for example, comprise two amino acid sequences having at least about 60% homology to SEQ ID NO: 24 and/or two amino acid sequences having at least about 60% homology to SEQ ID NO: 25 and/or two amino acid sequences having at least about 60% homology to SEQ ID NO: 26. For example, the antibodies or antibody fragments may comprise two amino acid sequences having at least about 60% homology to SEQ ID NO: 24 and two amino acid sequences having at least about 60% homology to SEQ ID NO: 25 and two amino acid sequences having at least about 60% homology to SEQ ID NO: 26. For example, the antibodies or antibody fragments may comprise two amino acid sequences of SEQ ID NO: 24 and/or two amino acid sequences of SEQ ID NO: 25 and/or two amino acid sequences of SEQ ID NO: 26. For example, the antibodies or antibody fragments may comprise two amino acid sequences of SEQ ID NO: 24 and two amino acid sequences of SEQ ID NO: 25 and two amino acid sequences of SEQ ID NO: 26.

The antibodies or antibody fragments may, for example, comprise two amino acid sequences having up to 3 amino acid differences to SEQ ID NO: 24 and/or two amino acid sequences having up to 3 amino acid differences to SEQ ID NO: 25 and/or two amino acid sequences having up to 3 amino acid differences to SEQ ID NO: 26. For example, the antibodies or antibody fragments may comprise two amino acid sequences having up to 3 amino acid differences to SEQ ID NO: 24 and two amino acid sequences having up to 3 amino acid differences to SEQ ID NO: 25 and two amino acid sequences having up to 3 amino acid differences to SEQ ID NO: 26. For example, the antibodies or antibody fragments may comprise two amino acid sequences of SEQ ID NO: 24 and/or two amino acid sequences of SEQ ID NO: 25 and/or two amino acid sequences of SEQ ID NO: 26. For example, the antibodies or antibody fragments may comprise two amino acid sequences of SEQ ID NO: 24 and two amino acid sequences of SEQ ID NO: 25 and two amino acid sequences of SEQ ID NO: 26.

In certain embodiments, the amino acid sequences having at least about 60% homology to SEQ ID NO: 24 or SEQ ID NO: 25 or SEQ ID NO: 26 each independently have a homology of at least about 65% or at least about 70% or at least about 75% or at least about 80% or at least about 85% or at least about 90% or at least about 95% or at least about 98% or at least about 99%. For example, the amino acid sequences having at least about 60% homology to SEQ ID NO: 24 or SEQ ID NO: 25 or SEQ ID NO: 26 may each independently have up a homology up to about 100%. In certain embodiments, the amino acid sequences having at least about 60% homology to SEQ ID NO: 24 or SEQ ID NO: 25, or SEQ ID NO: 26 each have a homology of at least about 80%.

In certain embodiments, the amino acid sequences having up to 3 amino acid differences to SEQ ID NO: 24 or SEQ ID NO: 25 or SEQ ID NO: 26 each independently have up to 2 amino acid differences or up to 1 amino acid differences to SEQ ID NO: 24 or SEQ ID NO: 25 or SEQ ID NO: 26. For example, the amino acid sequences having up to 3 amino acid differences to SEQ ID NO: 24 or SEQ ID NO: 25 or SEQ ID NO: 26 may each independently have 0 amino acid differences to SEQ ID NO: 24 or SEQ ID NO: 25 or SEQ ID NO: 26. In certain embodiments, the amino acid sequences having up to 3 amino acid differences to SEQ ID NO: 24 or SEQ ID NO: 25 or SEQ ID NO: 26 each have up to 2 amino acid differences or up to 1 amino acid difference to SEQ ID NO: 24, SEQ ID NO: 25, or SEQ ID NO: 26.

The antibody or antibody fragment may, for example, comprise an amino acid sequence having at least about 60% homology to SEQ ID NO: 19. For example, the antibody or antibody fragment may comprise an amino acid sequence having at least about 65% or at least about 70% or at least about 75% or at least about 80% or at least about 85% or at least about 90% or at least about 95% or at least about 98% or at least about 99% to SEQ ID NO: 19. For example, the antibody or antibody fragment may comprise an amino acid sequence having up to 100% homology to SEQ ID NO: 19. For example, the antibody or antibody fragment may comprise SEQ ID NO: 19. The antibody or antibody fragment may, for example, comprise two amino acid sequences having at least about 60% homology to SEQ ID NO: 19. For example, the antibody or antibody fragment may comprise two amino acid sequences of SEQ ID NO: 19.

SEQ ID NO: 19 or the sequence having at least about 60% homology thereto may, for example, occur on the heavy chain of the antibody or antibody fragment. SEQ ID NO: 19 may, for example, form part or all of a variable domain of the antibody or antibody fragment.

The antibody or antibody fragment may, for example, comprise an amino acid sequence having at least about 60% homology to SEQ ID NO: 23. For example, the antibody or antibody fragment may comprise an amino acid sequence having at least about 65% or at least about 70% or at least about 75% or at least about 80% or at least about 85% or at least about 90% or at least about 95% or at least about 98% or at least about 99% to SEQ ID NO: 23. For example, the antibody or antibody fragment may comprise an amino acid sequence having up to 100% homology to SEQ ID NO: 23. For example, the antibody or antibody fragment may comprise SEQ ID NO: 23. The antibody or antibody fragment may, for example, comprise two amino acid sequences having at least about 60% homology to SEQ ID NO: 23. For example, the antibody or antibody fragment may comprise two amino acid sequences of SEQ ID NO: 23.

SEQ ID NO: 23 or the sequence having at least about 60% homology thereto may, for example, occur on the light chain of the antibody or antibody fragment. SEQ ID NO: 23 may, for example, form part of a variable domain of the antibody or antibody fragment.

The antibody or antibody fragment may, for example, comprise one or more constant domains, for example on a light chain or a heavy chain. The constant domains may, for example, be present in a Fab and/or a Fc region of the antibody or antibody fragment. The constant domains may, for example, be IgA constant domains or IgD constant domains or IgE constant domains or IgG constant domains or IgM constant domains. For example, the constant domains may be IgG constant domains.

In a further aspect of the present invention there is provided an antibody obtained from or obtainable from the hybridoma having the ECACC accession number 19041601. As described in the examples below, these antibodies bind complement protein C7 in isolation or as part of a protein complex and inhibit complement activation.

In a further aspect of the present invention there is provided an antibody obtained from or obtainable from the hybridoma having the ECACC accession number 19080601. As described in the examples below, these antibodies bind complement protein C7 in isolation or as part of a protein complex and inhibit complement activation.

In a further aspect of the present invention there is provided an antibody obtained from or obtainable from the hybridoma having the ECACC accession number 19080602. As described in the examples below, these antibodies bind complement protein C7 in isolation or as part of a protein complex and inhibit complement activation.

In a further aspect of the present invention there is provided an antibody obtained from or obtainable from the hybridoma having the ECACC accession number 19080603. As described in the examples below, these antibodies bind complement protein C7 in isolation or as part of a protein complex and inhibit complement activation.

The antibody or antibody fragment may, for example, be a humanized antibody or antibody fragment. Humanized antibodies or antibody fragments are antibodies or antibody fragments from non-human species that have been modified to increase their similarity to antibodies produced naturally by humans, thereby reducing risk of immunogenicity. For example, the antibody or antibody fragment may have been altered such that at least about 80% of amino acid sequences other than the complementarity determining regions (CDRs) are identical to a human antibody or antibody fragment. For example, at least about 85% or at least about 90% or at least about 91% or at least about 92% or at least about 93% or at least about 94% or at least about 95% or at least about 96% or at least about 97% or at least about 98% or at least about 99% of amino acid sequences other than the complementarity determining regions (CDRs) are identical to a human antibody or antibody fragment. For example, up to 100% of amino acid sequences other than the complementarity determining regions (CDRs) are identical to a human antibody or antibody fragment. In certain embodiments, the antibody or antibody fragment is a humanized version of the antibody obtained from or obtainable from the hybridoma having the ECACC accession number 19041601 or the hybridoma having the ECACC accession number 19080601 or the hybridoma having the ECACC accession number 19080602 or the hybridoma having the ECACC accession number 19080603.

The antibody or antibody fragment may, for example, be a chimeric antibody or antibody fragment. Chimeric antibodies or antibody fragments are antibodies or antibody fragments containing amino acid sequences from two or more species. Chimeric antibodies or antibody fragments are often made to make the antibody or antibody fragment more like a human antibody or antibody fragment but do not always fall within the definition of humanized antibodies or antibody fragments. For example, the antibodies and antibody fragments may have a constant region from a human antibody and a variable region from a non-human antibody (e.g. a mouse or rat antibody). In certain embodiments, the antibody or antibody fragment is a chimeric version of the antibody obtained from or obtainable from the hybridoma having the ECACC accession number 19041601 or the hybridoma having the ECACC accession number 19080601 or the hybridoma having the ECACC accession number 19080602 or the hybridoma having the ECACC accession number 19080603.

The antibody or antibody fragment may, for example, be an affinity-matured antibody or antibody fragment. Affinity-matured antibodies or antibody fragments are antibodies or antibody fragments that have undergone a process to mutate amino acids in one or more CDRs of the antibody or antibody fragment in order to increase affinity to the antigen (e.g. complement protein C7 in isolation or as part of a protein complex) or to introduce novel properties (e.g. pH switch properties). Such mutations may be randomly introduced using radiation, chemical mutagens, or error-prone PCR, or may be specifically engineered using molecular methods. In certain embodiments, the antibody or antibody fragment is an affinity-matured version of the antibody obtained from or obtainable from the hybridoma having the ECACC accession number 19041601 or the hybridoma having the ECACC accession number 19080601 or the hybridoma having the ECACC accession number 19080602 or the hybridoma having the ECACC accession number 19080603.

The antibody or antibody fragment may, for example, be a recombinant antibody or antibody fragment. Recombinant antibodies or antibody fragments are antibodies or antibody fragments that have been produced using recombinant antibody coding genes. In certain embodiments, the antibody or antibody fragment is a recombinant version of the antibody obtained from or obtainable from the hybridoma having the ECACC accession number 19041601 or the hybridoma having the ECACC accession number 19080601 or the hybridoma having the ECACC accession number 19080602 or the hybridoma having the ECACC accession number 19080603.

The antibody or antibody fragment may, for example, be modified to include one or more labels. The labels may, for example, be radioactive labels, fluorescent labels, luminescent labels, biotin labels, and enzymatic labels.

The antibodies and antibody fragments may be produced by an in vitro or in vivo method known to those skilled in the art, for example using recombinant technology and/or hybridoma technology. For example, the antibodies or antibody fragments may be produced by adding one or more amino acid sequences described herein to an existing antibody or antibody fragment using recombinant nucleic acid technology or replacing one or more amino acid sequences with one or more amino acid sequences described herein. For example, the antibodies or antibody fragments may be produced by expressing the recombinant antibody or antibody fragment from a host cell as described herein.

In accordance with a further aspect of the present invention there is provided a pharmaceutical composition comprising one or more antibodies or antibody fragments described herein and a pharmaceutically-acceptable carrier, excipient, or diluent.

The antibodies or antibody fragments present in the pharmaceutical composition may, for example, be monoclonal (bind to the same epitope of C7). All the antibodies or antibody fragments present in the pharmaceutical composition may, for example, be the same.

The term “pharmaceutical composition” refers to a composition comprising (a pharmaceutically effective amount of) an antibody or antibody fragment as described herein and additionally one or more pharmaceutically acceptable carriers and/or excipients and/or diluents. The pharmaceutical composition may further contain ingredients selected from, for example, diluents, adjuvants, excipients, vehicles, preserving agents, fillers, binders, disintegrating agents, wetting agents, emulsifying agents, suspending agents, sweetening agents, flavouring agents, perfuming agents, antibacterial agents, antifungal agents, lubricating agents, coating agents, encapsulating agents and dispersing agents, depending on the nature of the mode of administration and dosage forms. The pharmaceutical compositions may take the form, for example, of solid preparations including tablets, capsules, caplets, dragees, lozenges, granules, powders, pellets, beads and cachets; and liquid preparations including elixirs, syrups, suspensions, sprays, emulsions, lotions, creams and solutions. Techniques and formulations generally may be found in Remington, The Science and Practice of Pharmacy, Mack Publishing Co., Easton, Pa., latest edition.

In certain embodiments, the pharmaceutical composition further comprises one or more additional therapeutic agents. For example, the one or more additional therapeutic agents may be therapeutic agent(s) useful for the treatment or prevention of a disease associated with dysregulation of complement.

The pharmaceutical composition may, for example, comprise at least about 30 wt % of the antibody or antibody fragment described herein. For example, the pharmaceutical composition may comprise at least about 35 wt % or at least about 40 wt % or at least about 45 wt % or at least about 50 wt % or at least about 55 wt % or at least about 60 wt % or at least about 65 wt % or at least about 70 wt % of the antibody or antibody fragment described herein. For example, the pharmaceutical composition may comprise up to about 95 wt % of the antibody or antibody fragment described herein. For example, the pharmaceutical composition may comprise up to about 90 wt % or up to about 85 wt % or up to about 80 wt % or up to about 75 wt % or up to about 70 wt % of the antibody or antibody fragment described herein. For example, the pharmaceutical composition may comprise from about 30 wt % to about 90 wt % or from about 40 wt % to about 80 wt % or from about 50 wt % to about 70 wt % of the antibody or antibody fragment described herein.

The pharmaceutical composition may, for example, comprise at least about 5 wt % total pharmaceutically-acceptable excipient and carrier and diluent. For example, the pharmaceutical composition may comprise at least about 10 wt % or at least about 15 wt % or at least about 20 wt % or at least about 25 wt % or at least about 30 wt % total pharmaceutically-acceptable excipient and carrier and diluent. For example, the pharmaceutical composition may comprise up to about 70 wt % total pharmaceutically-acceptable excipient and carrier and diluent. For example, the pharmaceutical composition may comprise up to about 65 wt % or up to about 60 wt % or up to about 55 wt % or up to about 50 wt % or up to about 45 wt % or up to about 40 wt % total pharmaceutically-acceptable excipient and carrier and diluent. For example, the pharmaceutical composition may comprise from about 5 wt % to about 70 wt % or from about 10 wt % to about 60 wt % or from about 15 wt % to about 50 wt % total pharmaceutically-acceptable excipient and carrier and diluent.

Liquid form preparations include solutions, suspensions, and emulsions, for example, water or water-propylene glycol solutions for oral administration. Liquid preparations can also be formulated in solution in aqueous polyethylene glycol solution. In certain embodiments, the active ingredient(s), i.e., antibodies or antibody fragments, may be mixed with one or more pharmaceutically acceptable carriers and/or excipients and/or diluents, such as water and/or any of the following: solvent such as propylene glycol, alcohol; humectant such as glycerol; sweeteners such as liquid glucose, corn syrup and sucrose; artificial sweeteners such as aspartame, stevia and sucralose; preservatives such as benzoates and parabens; viscosity modifiers/thickeners such as gums and alginates; buffering agents; flavouring agents and colouring agents.

The pharmaceutical composition may, for example, be a pharmaceutical composition (medicament), suitable for oral, nasal, topical, suppository, intravenous, subcutaneous or intradermal administration.

In a further aspect of the present invention there is provided a nucleic acid sequence encoding an antibody or antibody fragment of the present invention. The nucleic acid sequence may, for example, be a DNA or RNA molecule.

The nucleic acid sequence may, for example, be part of a vector comprising a nucleic acid sequence encoding an antibody or antibody fragment of the present invention operably linked to a nucleic acid sequence that promotes expression of the antibody or antibody fragment of the present invention. The vector may, for example, be a plasmid (a circular double stranded DNA loop into which additional DNA segments may be ligated). Alternatively, the vector may be a viral vector wherein additional DNA segments may be inserted into the viral genome. Certain vectors are capable of autonomous replication in a host cell into which they are introduced (e.g. bacterial vectors having a bacterial origin of replication and episomal mammalian vectors). Other vectors (e.g. non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

In a further aspect of the present invention there is provided a cell comprising a nucleic acid sequence encoding an antibody or antibody fragment of the present invention. The cell may, for example, comprise two or more nucleic acid sequences encoding an antibody or antibody fragment of the present invention, which may, for example, be the same or different. The cell may, for example, be referred to as a recombinant and/or host cell. The cell may, for example, be an established cell line (a cell that demonstrates the potential for indefinite subculture in vitro).

In a further aspect of the present invention there is provided a cell secreting an antibody or antibody fragment of the present invention. The cell may, for example, comprise one or more nucleic acid sequence(s) encoding an antibody or antibody fragment of the present invention. The cell may, for example, be referred to as a recombinant and/or host cell. The cell may, for example, be an established cell line (a cell that demonstrates the potential for indefinite subculture in vitro).

The cell may, for example, be a hybridoma. In certain embodiments, the hybridoma has the ECACC accession number 19041601 or the ECACC accession number 19080601 or the ECACC accession number 19080602 or the ECACC accession number 19080603.

The cell may, for example, be prokaryotic (e.g. Escherichia coli) or eukaryotic (e.g. protist cell, animal cell (e.g. mammalian cell such as CHO or COS or HEK 293 cells, avian cell, insect cell such as Sf9 cell), plant cell, fungal cell (e.g. yeast cell such as Saccharomyces cerevisiae)).

In accordance with a further aspect of the present invention there is provided the therapeutic and non-therapeutic (e.g. in vitro) uses of the antibodies and antibody fragments of the present invention.

For example, the antibodies and antibody fragments of the invention may be used for diagnosing, detecting, monitoring, treating or preventing a disease. For example, the antibodies and antibody fragments of the invention may be used to detect C7 (in isolation or as part of a protein complex) in a sample obtained from a subject.

The expression “treating or preventing” and analogous terms used herein refers to all forms of healthcare intended to remove or avoid the disease or to relieve its symptoms, including preventive and curative care, as judged according to any of the tests available according to the prevailing medical practice. An intervention that aims with reasonable expectation to achieve a particular result but does not always do so is included within the expression “treating or preventing”. An intervention that succeeds in slowing or halting progression of a disease is included within the expression “treating or preventing”.

The disease may, for example, be a disease associated with the dysregulation of complement, i.e. a disease caused by or resulting in abnormal complement activity in a subject. For example, the disease may be caused by or result in an excessive level of complement activity. For example, the disease may be caused by or result in activation of complement by an inappropriate trigger. For example, the disease may be a type II or type III hypersensitivity disease. For example, the disease may be an autoimmune disease. For example, the disease may be an autoinflammatory disease. For example, the disease may be an iatrogenic disease. For example, the disease may be a degenerative disease.

Examples of diseases associated with the dysregulation of complement include paroxysmal nocturnal hemoglobinuria (PNH), atypical haemolytic uremic syndrome (aHUS), hereditary angioedema (HAE), mismatched blood transfusions, blood compatibility diseases (e.g. erythroblastosis fetalis), systemic lupus erythematosus (SLE), C3 glomerulopathy (C3G), age-related macular degeneration (AMD), myasthenia gravis, neuromyelitis optica, multiple sclerosis (MS), rheumatoid arthritis (RA), Hidradenitis suppuritiva, polyarteritis, inflammatory effects of cardiopulmonary bypass and haemodialysis, transplant rejection, (ANCA)-associated vasculitis, Alzheimer's disease, atypical haemolytic uremic syndrome, acute kidney injury, antibody-mediated rejection, antiphospholipid syndrome, acute respiratory distress syndrome, Berger's disease, bullous pemphigoid, cold agglutinin disease, chronic obstructive pulmonary disease, cardiopulmonary bypass, dense deposit disease, delayed graft function, geographic atrophy, Guillain-Barre syndrome, refractory generalized myasthenia gravis, granulomatosis with polyangiitis, graft versus host disease, hereditary angioedema, IgA nephropathy, hematopoietic stem cell transplant-related TMA, ischemia/reperfusion injury, immune complex membranoproliferative glomerulonephritis, idiopathic polypoidal choroidal vasculopathy, kidney transplant, lupus nephritis, membranous nephropathy, microscopic polyangiitis, neuromyelitis optical spectrum disorder, rheumatoid arthritis/osteoarthritis, systemic inflammatory response syndrome, systemic lupus erythematosus, Stargardt disease 1, sepsis, severe sepsis, septic shock, thrombotic microangiopathy, warm type autoimmune haemolytic anemia, wet AMD, periodontitis, asthma, Crohn's disease, atherosclerosis, vasculitis, hemodialysis, psoriasis, burns, myocardial infarction, glaucoma, uveitis, trauma, Alzheimer's disease, and Parkinson's disease. Preferred diseases are autoimmune, inflammatory and degenerative diseases of current unmet need, including primary progressive multiple sclerosis, refractory rheumatoid arthritis, and neurodegenerative diseases.

In certain embodiments, the subject is a human. In other embodiments, the subject is a mammal other than a human, such as non-human primates (e.g. apes, monkeys and lemurs), companion animals such as cats or dogs, working and sporting animals such as dogs, horses and ponies, farm animals such as pigs, sheep, goats, deer, oxen and cattle, and laboratory animals such as rodents (e.g. rabbits, rats, mice, hamsters, gerbils or guinea pigs).

The antibody or antibody fragment or pharmaceutical composition or nucleic acid sequence may be administered to the subject. For example, the nucleic acid sequence may be administered to the subject such that the antibody or antibody fragment is expressed by one or more cells in the subject.

The antibody or antibody fragment or pharmaceutical composition or nucleic acid sequence may, for example, be administered to the subject orally, nasally, topically, as a suppository, intravenously, intrathecally, subcutaneously, or intradermally. For example, the antibody or antibody fragment or pharmaceutical composition or nucleic acid sequence may be administered to the subject intravenously.

The amount of antibody or antibody fragment administered may be varied depending on the requirements of the subject, the desired results, and the severity of the condition being treated. Determination of the proper amount/dosage for a particular situation is within the skill of the art. For example, for therapeutic applications a physician or veterinarian having ordinary skill in the art can readily determine and prescribe the effective amount of the pharmaceutical composition required. The total daily amount/dosage may be divided and administered in portions during the day if desired.

The invention will now be described in detail by way of reference only to the following non-limiting examples.

### EXAMPLES

### Example 1

Preparation of Antibodies

Monoclonal antibodies against C7 protein were generated by immunizing wild-type and/or C7-deficient mice with pure C7 protein and isolating hybridoma clones using classical cloning methods. These mice were also used as a source of macrophages as a feeding support for hybridoma cell lines. Hybridoma clone supernatants were screened for binding C7 by ELISA and for inhibition of complement by high throughput haemolytic assay. The anti-C7 antibodies 17E7, 59E7, 3B11, 2H2, and 73D1 were selected for further testing. The hybridoma that produces 17E7 was deposited under the ECACC accession number 19041601 on 16 Apr. 2019. The hybridoma that produces 59E7 was deposited under the ECACC accession number 19080602 on 6 Aug. 2019. The hybridoma that produces 73D1 was deposited under the ECACC accession number 19080601 on 6 Aug. 2019. The 17E7 antibody was made in wild-type mice whereas the other antibodies were made in C7-deficient mice. The hybridoma that produces 2H2 was deposited under the ECACC accession number 19080603 on 6 Aug. 2019.

Monoclonal anti-C5 antibodies were also generated in-house by the same methods but immunising C5-deficient mice with pure C5, to use as controls. The control antibodies used were as follows.

7D4: Human/rat specificity

BB5.1: Mouse specificity

The commercially available anti-C5 antibodies Eculizumab (obtained from Alexion), Crovalimab (original name SKY59, obtained from Roche) were used as controls. Eculizumab is specific to human C5. Crovalimab (SKY59) is specific to human C5 and weakly specific to mouse C5.

Test Methods

Direct Enzyme Linked Immunosorbent Assay (ELISA)

Maxisorp 96 well plates were coated with human, monkey (cyno), rat or mouse C7 protein at final concentration of 0.5 μg/ml. Wells were blocked with bovine serum albumin (BSA), washed, then incubated (1 hour at 37° C.) with test samples containing mAb (supernatants from hybridoma clones for example). Wells were washed then incubated as above with peroxidase-labelled anti-mouse immunoglobulin (Jackson Labs). Wells were washed and signals were developed with OPD (o-phenylenediamine dihydrochloride; Sigma). Colour development was stopped by adding 10% H₂SO₄ to all wells, usually within 5 minutes. The absorbance of each well was measured at 492 nm. Cells from wells with the greatest colour development were taken forward.

Standard Classical Pathway Haemolysis Assays (CP)

The assay system comprises optimally antibody sensitised sheep erythrocytes (ShEA) incubated with normal human serum (NHS), normal rat serum (NRS), normal male mouse serum (NMS) and normal rabbit serum (NRbS) dilutions in complement fixation diluent (CFD). The serum dilution was selected to give ˜75-90% haemolysis at end point (30 minutes at 37° C.; typically 2-5% serum, titrated before assay) in the absence of inhibitor. For measurement of cP activity in NMS, ShEA were additionally incubated with mouse anti-rabbit IgG at 25 μg/ml (#3123; Invitrogen) for 30 minutes at 37° C. before washing in HBS. Test and control mAb are titrated in a broad range of concentrations (in triplicate) into the serum dilutions (100 μl) in the wells of a U-welled microtitre plate and ShEA (100 μl) at the appropriate dilution in CFD added and incubated for 30 minutes at 37° C. Plates are centrifuged, supernatants removed to a flat-welled microtitre plate and absorbances measured spectrophometrically. % haemolysis is calculated for each well by comparison with 0 and 100% lysis controls.

Serum-Excess Classical Pathway Haemolysis Assays (SE-CP)

This assay was used to better reflect conditions prevailing in whole blood. The assay system comprises optimally antibody sensitised sheep erythrocytes (ShEA) incubated with a NHS or NRS concentration 10-fold that used in the standard assay above (20-50%). Test and control mAb are titrated in a broad range of concentrations (in triplicate) into the NHS or NRS dilutions (100 μl) in the wells of a U-welled microtitre plate and ShEA (100 μl) at the appropriate dilution in CFD added and incubated for 30 minutes at 37° C. Plates are centrifuged, supernatants removed to a flat-welled microtitre plate and absorbances measured spectrophometrically. % haemolysis is calculated for each well by comparison with 0 and 100% lysis controls.

Reactive Lysis Assay System (RL)

The assay system comprises unsensitised guinea pig erythrocytes (GpE) in CFD incubated sequentially with purified C5b6, C7, C8 and C9; C5b6 dose is titrated to give ˜75-90% haemolysis after 15 minutes at 37° C. in the absence of inhibitor. All other components are in excess. Test and control mAb are titrated in a broad range of concentrations (in triplicate) into the wells, either prior to addition of C5b6, prior to addition of C7, or prior to addition of C8/C9 and incubated for 30 minutes at 37° C. Plates are centrifuged, supernatants removed to a flat-welled microtitre plate and absorbances measured spectrophometrically. % haemolysis is calculated for each well by comparison with 0 and 100% lysis controls.

Western Blotting (WB)

Western blot analysis was used to confirm the specificity of selected mAb for human, monkey, rat and mouse C7 protein. C7 protein or diluted human or animal sera was separated on SDS-PAGE, and proteins from the gel transferred to nitrocellulose by electroblotting. Nitrocellulose blots were blocked with 5% BSA, washed and incubated with dilutions of the test mAb for 1 hour at room temperature. The blots were washed then incubated with horseradish peroxidase HRP-conjugated anti-mouse immunoglobulin for 1 hour at room temperature with constant mixing. Blots were washed as described above and HRP-labelled bands were detected with enhanced chemiluminescence ECL (Pierce) and visualised by autoradiography. Molecular weights were calculated by comparison with protein standards run on the same gels/blots.

High-Throughput Classical Pathway Haemolysis Assay for Monoclonal Antibody Clones Screening

The assay system comprises an appropriate dilution of ShEA incubated with a NHS dilution in CFD titrated to give ˜75-90% haemolysis after 30 minutes at 37° C. (typically 2-5% NHS) in the absence of inhibitor. To determine the appropriate dilution of clone supernatant for the assay, fresh medium (F15) in which the fused hybridoma clones are cultured is titrated in the assay. The maximum non-inhibitory dilution of medium dictates the dilution of clone supernatant used for screening. Culture supernatants from individual wells are then added into the NHS dilutions (100 μl) in the wells of a U-welled microtitre plate and ShEA (100 μl) at the appropriate dilution in CFD added and incubated for 30 minutes at 37° C. Plates are centrifuged, supernatants removed to a flat-welled microtitre plate and absorbances measured spectrophometrically. % haemolysis is calculated for each well by comparison with 0 and 100% lysis controls.

Measuring Kinetics and Affinity of the Selected Anti-C7 mAbs

Biacore analyses were used to examine the characteristics of antibody-antigen interaction. Biacore uses Surface Plasmon Resonance (SPR) technology for the label-free, real time measurement of association (on) & dissociation (off) rates (ka & kd) and calculated binding affinity constant (KD) of the antibody-antigen interaction. Incorporating Biacore scouting assays early in the antibody screening process helps selection and ranking of the mAb based on their binding kinetics. This allowed early selection of the right antibodies for further characterisation. In addition, Biacore SPR technology can also identify mAb that bind distinct and separate epitopes on the antigen which added value for assay development.

In Vivo Complement Inhibition by mAb 17E7 and 73D1

A small animal study was conducted to demonstrate the capacity of mAb 17E7 and 73D1 to inhibit the complement system in vivo.

To test clone 73D1, wild-type mice were pre-bled, then the test group animals were injected subcutaneously with 73D1 mAb (1 mg), while control group mice were injected with BB5.1 anti-C5 mAb (1 mg). All mice were bled after a further two hours, again at 24 hours and finally at 48 hours. Serum from each of the bleeds (pre-bleed, 2, 24 and 48 hours post mAb administration) was tested in a classical pathway haemolysis assay as described herein and % haemolysis calculated for each sample. The results are shown in FIG. 30.

To test clone 17E7, (human-specific) C7-deficient mice (10) were pre-bled, then injected intraperitoneally with human C7 (500 μg), then split into test and control groups (5 in each). After 1 hour, all mice were test bled, then the test group animals were injected subcutaneously with 17E7 mAb (1 mg), while control group mice were injected with an irrelevant mAb (1 mg). All mice were bled after a further two hours. Serum from each of the bleeds (pre-bleed, post C7 administration, 2 hours post mAb administration) was tested in a classical pathway haemolysis assay as described herein and % haemolysis calculated for each sample.

Antibody Complementarity-Determining Regions (CDR) Sequence Determination

The antibody sequence through the CDRs was obtained under contract by Absolute Antibody. The complementarity determining regions (CDRs) were automatically identified on the “CDR identification” worksheet using Excel formulas working to the Kabat definition for CDRs. CDR identification was only performed for the primary VH and VL sequences.

Direct Enzyme Linked Immunosorbent Assay (ELISA) to Test Cross-Reactivity with Non-Human Primate C7

Maxisorp 96 well plates were coated with cynomolgus (cyno) monkey C7 protein at a final concentration of 0.5 μg/ml. Wells were blocked with bovine serum albumin (BSA), washed, then incubated (1 hour at 37° C.) with test samples containing mAb (supernatants from clones for example). Wells were washed then incubated as above with peroxidase-labelled anti-mouse immunoglobulin (Jackson Labs). Wells were washed and signals developed with OPD (o-phenylenediamine dihydrochloride; Sigma). Colour development was stopped by adding 10% H₂SO₄ to all wells, usually within 5 minutes. The absorbance of each well was measured at 492 nm.

Dot Blot to Test Binding of C7-FIMs by the Selected Anti-C7 mAb

Dot blot analysis was used to confirm specificity of selected mAb for C7-FIMs (Factor I modules of C7 protein; recombinant protein produced in house). FIMs are involved in the early stages of MAC formation and are likely candidates for blocking mAb binding sites. C7-FIMs protein (crude extract) was dotted (5 μl) onto nitrocellulose membrane and dried for 30 minutes. Nitrocellulose was blocked with 5% BSA, washed and incubated with dilutions of 17E7 mAb or Polyclonal Goat anti-human C7 (poly) (positive control) for 1 hour at room temperature. The blots were washed then incubated with HRP-conjugated secondary antibodies (mouse or goat), washed and HRP-label detected with ECL (Pierce) and visualised by autoradiography.

Sandwich ELISA to Test Epitope Competition for Binding to Human C7

Sandwich ELISA with polyclonal (Goat anti-C7) antibody (poly) as capture were used to confirm C7 binders in order to eliminate issues around denaturation of C7 protein in direct binding assays; similar assays using the different mAbs individually as capture and testing whether the various mAbs bound the captured C7 (indicating distinct epitopes) or failed to bind (indicating shared epitopes). Maxisorp plates were coated with the mAb (2 μg/ml in bicarbonate buffer, pH 9.6) at 4° overnight; wells were blocked (1 hour at 37° C. with 2% BSA-PBS) and washed in PBS-T. Standard curves of in-house purified human C7 protein diluted in 0.2% BSA-PBS, were added in triplicate and incubated for 1 hour at 37° C., followed after washing with peroxidase-labelled mAb (in house labelled, 1:1000 dilution in 0.2% BSA-PBS). Wells were washed and signals developed with OPD (o-phenylenediamine dihydrochloride; Sigma). Colour development was stopped by adding 10% H₂SO₄ to all wells, usually within 5 minutes. The absorbance of each well was measured at 492 nm.

Western Blot and ELISA to Identify the Composition of the Pull-Down Complex

Complement in human or rat serum (˜3 ml) was activated via both classical and alternative pathways by incubation with Zymosan A (7 mg/ml) and aggregated human IgG (1 mg/ml) in the presence of biotinylated mAb 2H2, 17E7, 59E7 or 73D1 (in house). The mixture was incubated for 32 hours at 37° C. in a shaking water bath to ensure that all the C5 was converted into the complex. The reaction was stopped by centrifugation at 2500 rpm for 15 minutes at 4° C., the supernatant (activated serum) collected and mixed with 1 ml Avidin-coated beads. The mixture was incubated for one hour at ambient while mixing gently, then beads washed five times in PBS by centrifugation and the bound mAb 2H2, 17E7, 59E7 or 73D1-complex eluted by incubation (10 minutes at 100° C.) in reduced or non-reduced SDS-PAGE running buffer. Supernatants were subjected to SDS-PAGE and WB. The proteins were separated on SDS-PAGE and transferred to nitrocellulose by electroblotting. Nitrocellulose blots were blocked with 5% BSA, washed and incubated with dilutions of the Goat anti-C5, -C6, -C7, -C8 or -C9 antibodies (at 2 μg/ml) for 1 hour at room temperature. The blots were washed then incubated with horseradish peroxidase HRP-conjugated anti-goat immunoglobulin (1:10 000 dilutions, Jackson Immunoresearch) for 1 hour at room temperature with constant mixing. Blots were washed and HRP-labelled bands were detected with enhanced chemiluminescence ECL (Pierce) and visualised by autoradiography.

The complexes pulled down from activated serum with biotinylated mAbs 2H2, 17E7, 59E7 or 73D1 were also tested in ELISA. Briefly, plates were coated with Avidin (10 μg/ml); after BSA blocking, the pull-down samples were added and detected with Goat anti-C5, 06, 07, 08 or C9 antibodies, then developed with rabbit anti-Goat-HRP. Controls for each antibody comprised biotinylated mAb alone or incubated with C7.

Results

ELISA

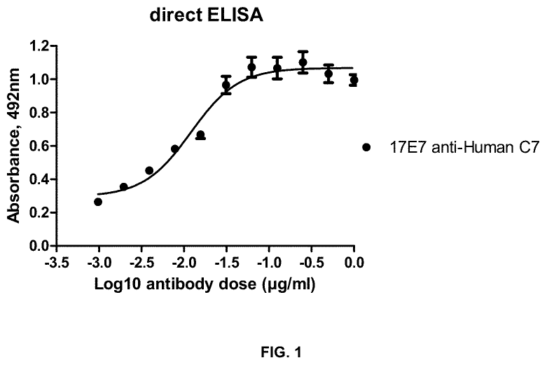

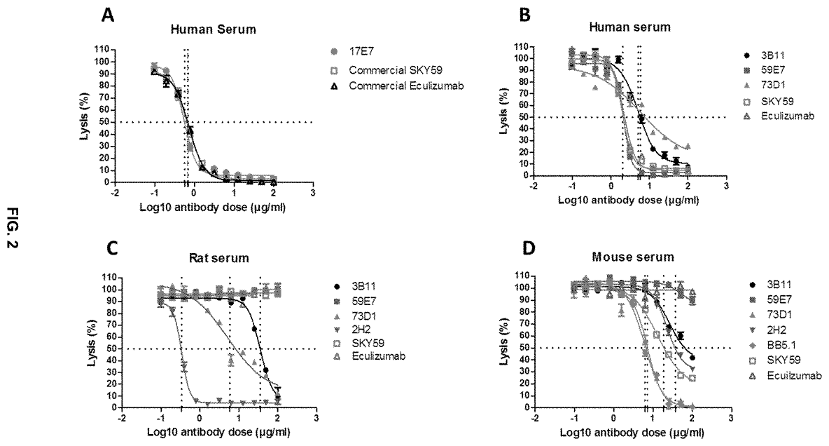

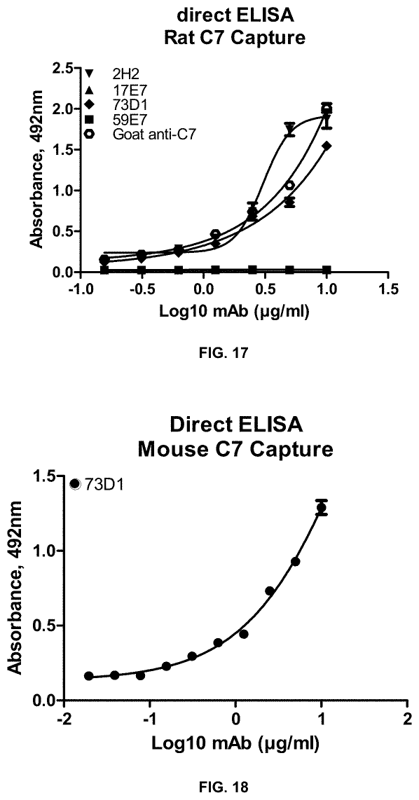

The results are shown in FIGS. 1 and 16 to 18. It was found that the mAb 17E7 binds C7 in direct ELISA. Further, all antibodies bind human and cyno C7. 2H2 and 73D1 bind rat C7. 73D1 binds mouse C7.

Standard Classical Pathway Haemolysis Assays (CP)

Assay Protocol: Standard classical pathway haemolysis assays (CP50)

mAb dilution: 100/50/25/12.5/6.3/3.1/1.6/0.8/0.39/0.20/0.10/0 μg/ml

Human, rat, serum dilution: 2.5%; rabbit, mouse: 25

Erythrocytes: 2% ShEA

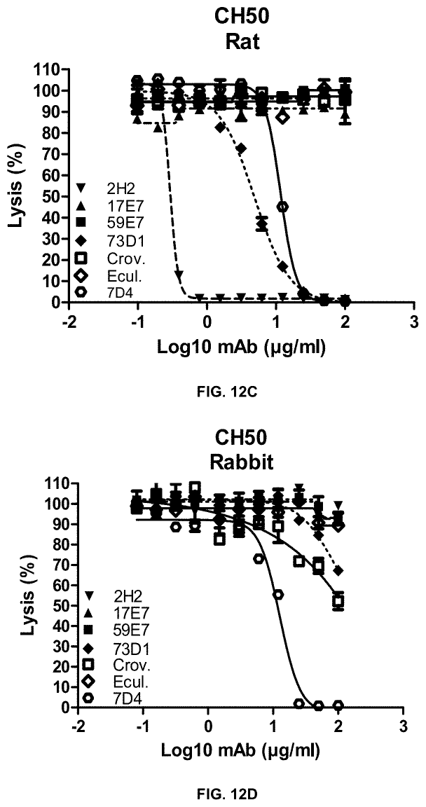

The results are shown in FIGS. 2 and 12 and Table 1 below.

TABLE 1

| | | | | Cross- | Cross- |
|---|---|---|---|---|---|
| | | | 50% | species | species |
| | | Haemolytic | Inhibitory | reactivity | reactivity |
| Antibody | Isotype | Units (HU) | Dose (ng/ml) | in vitro | in vivo |
| 17E7 | IgG2a, K | Human = 55.1 | Human = 181.6 | Strong; Human | Human |
| 59E7 | IgG2b, K | Human = 47.7 | Human = 209.7 | Strong; Human | Not tested |
| 3B11 | IgM, K | Human = 19.9 | Human = 501.9 | Strong; Human, Rat | Not tested |
| | | Rat = 11.9 | Rat = 839.5 | | |
| 2H2 | IgG2b, K | Human = 1.0 | Human = 9977.0 | Strong; Rat Weak; | Rat |
| | | Rat = 264.8 | Rat = 33.9 | Mouse, Human | |
| | | Mouse = 2.7 | Mouse = 3715.4 | | |
| 73D1 | IgG2a, K | Human = 17.2 | Human = 581.8 | Strong; Mouse, Weak; | Mouse |
| | | Rat = 19.8 | Rat = 505.5 | Human, Rat | |
| | | Mouse = 13.9 | Mouse = 720.3 | | |
| 7D4 | IgG2b, K | Human = 69.3 | Human = 144.2 | Strong; Human, Rat | Not tested |
| | | Rat = 9.5 | Rat = 1054.4 | | |
| BB5.1 | IgG1, K | Mouse = 16.3 | Mouse = 613.6 | Strong; Mouse | Not tested |
| SKY59 | IgG1, K | Human = 49.9 | Human = 212.3 | Strong; Human Weak; | Not tested |
| | | Mouse = 1.4 | Mouse = 1862.1 | mouse | |
| Eculizumab | IgG2/4, K | Human = 47.1 | Human = 181.6 | Strong; Human | Not tested |

It was found that the antibodies were efficient complement inhibitors for the species to which they are reactive and the dose required for inhibition of C7 is comparable to the dose required for inhibition of C5 for both of the control antibodies (SKY59 and Eculizumab).

17E7 and 59E7 are human specific. 2H2 and 73D1 cross-react with rat and mouse.

Serum Excess Classical Pathway Haemolysis Assays (Serum Excess CP)

Assay Protocol: Serum excess classical pathway haemolysis assays (Serum Excess CP)

mAb dilution: 1000/500/250/125/63/31/16/8/3.9/2/1/0 μg/ml

Serum dilution: 25

Erythrocytes: 2% ShEA

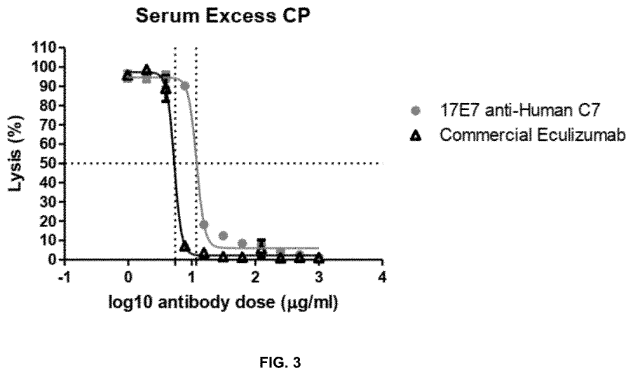

The results are shown in FIG. 3 and Table 1 above.

It was found that 17E7 inhibited lysis in this assay with 50% inhibitory dose and dose causing complete inhibition in the same range as the control Eculizumab. The higher doses needed here are expected for the increased serum dose.

Reactive Lysis Assay System (RL)

Assay Protocol: Reactive lysis assay system (RL)

mAb dilution: 1/0.5/0.25/0.125/0.063/0.032/0.016/0 μg/ml

Erythrocytes: 2% GpE

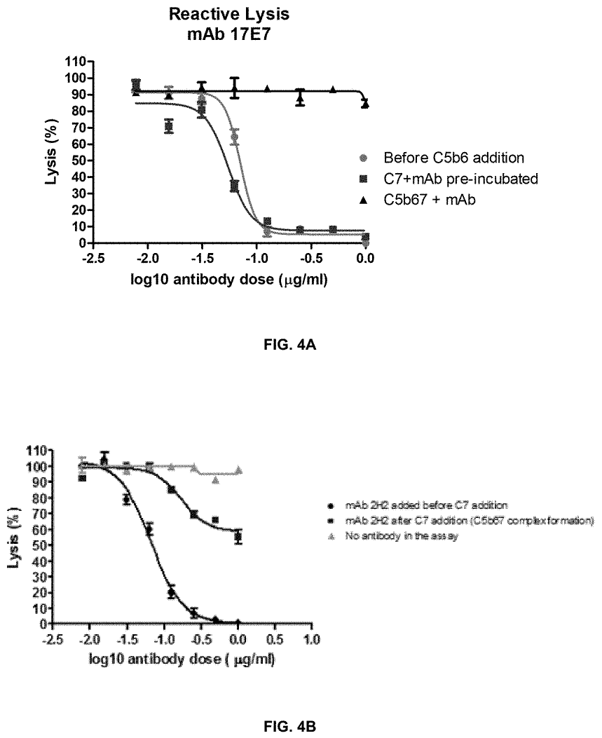

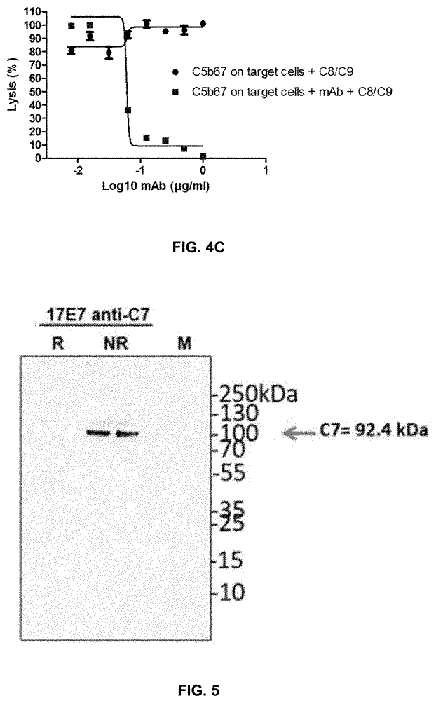

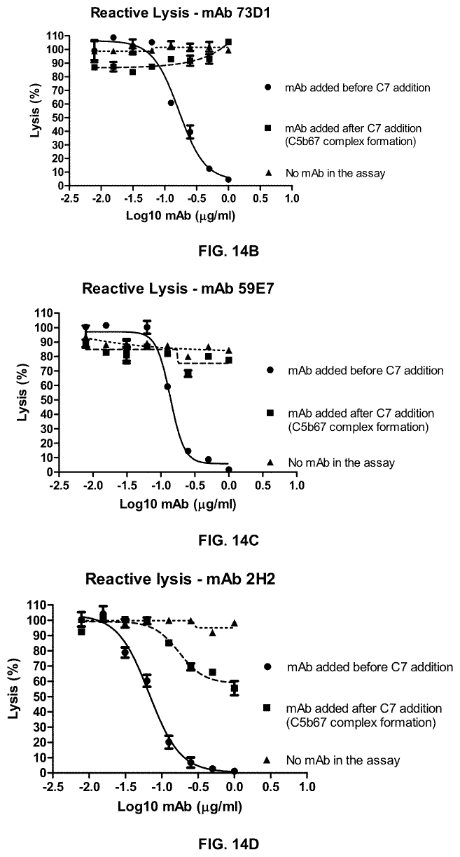

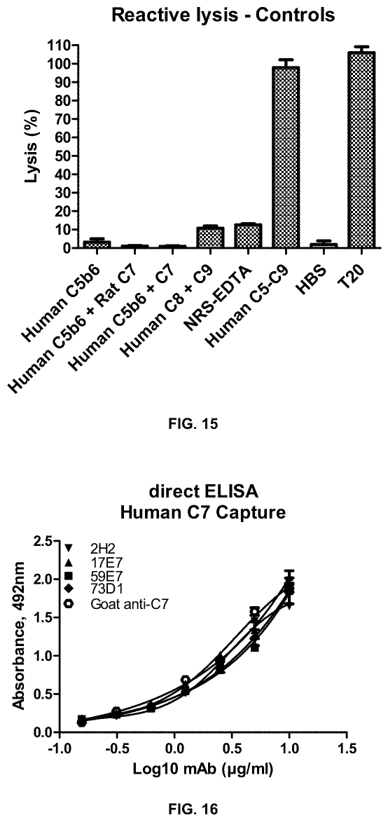

The results are shown in FIGS. 4, 14 and 15. It was found that 17E7 inhibited in the reactive lysis system when added before C5b6 or when added after C5b6 but preincubated with the C7 (FIG. 4A). The mAb did not inhibit when added after addition of C7 to form the C5b67 complex on the target cells. The data demonstrate that 17E7 blocks C7 binding to C5b6 but does not inhibit C8 binding to a pre-formed C5b67 complex. The mAb 2H2 inhibited when added either before or after addition of C7 to form the C5b67 complex (FIG. 4, A, B) or when the C5b67 complex was pre-formed on the target cells (FIG. 4C). The data demonstrate that 2H2 additionally inhibits C8 binding to a pre-formed C5b67 complex. All antibodies block C7 binding to C5b6 (FIG. 14). mAb 2H2 also weakly inhibits C8 binding to C5b67.

Western Blotting

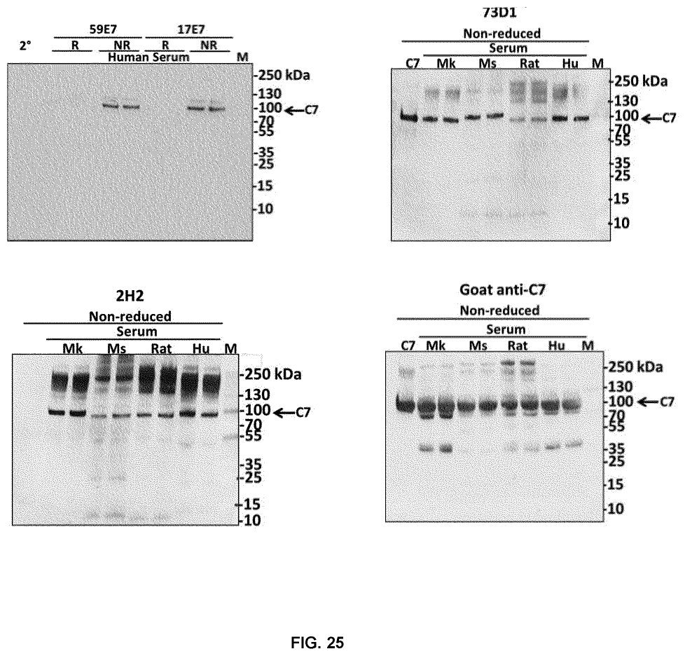

The results are shown in FIGS. 5 and 25. It was found that 17E7 detects C7 protein under non-reducing but not reducing conditions by western blot. All mAb bind human (Hu) C7. 2H2 and 73D1 also bind rat, mouse (Ms) and monkey (Mk) C7.

Kinetics and Affinity Check of the Monoclonal Anti-Human C7 Antibody Produced

The antibody was captured through Protein L or Fc on a Mouse IgG capture kit (attached to CM5 sensor chip) and human C7 dilutions (9, 17, 34, 68 μM) flowed over the surface for 120 seconds. Dissociation was then measured for a further 600 seconds. The surface was regenerated between each cycle.

The results are shown in Table 2 below.

TABLE 2

| | Ka | Kd | KD |
|---|---|---|---|
| Antibody | (1/Ms) | (1/s) | (M) |
| 17E7 | Human; | 6.94 × 10⁻⁵ | 1.02 × 10⁻⁹ |
| | 6.83 × 10⁴ | | |
| 59E7 | Human; | 1.4 × 10⁻³ | 9.31 × 10⁻¹⁰ |
| | 1.45 × 10⁶ | | |
| 3B11* | Human; | 1.20 × 10⁻³ | 2.30 × 10⁻⁷ |
| | 5.05 × 10³ | 5.61 × 10⁻⁴ | 1.93 × 10⁻⁷ |
| | Rat; | | |
| | 2.91 × 10³ | | |
| 73D1 | Human; | Human; | Human; |
| | 2.63 × 10⁴ | 1.5 × 10⁻³ | 5.55 × 10⁻⁸ |
| | Rat; | Rat; | Rat; |
| | 6.98 × 10³ | 5.70 × 10⁻⁴ | 8.17 × 10⁻⁸ |
| | Mouse; | | |
| | to be determined | | |
| 2H2 | Human; | to be determined | to be determined |
| | to be determined | | |
| | Rat; | | |
| | to be determined | | |

*Protein L capture

It was found that there is strong and stable binding of human C7 to the mAb 17E7. The mAb 17E7 has a high affinity for C7 and low off rate from C7. The mAb 59E7 also strongly binds human C7 albeit with a slightly faster off rate. The mAbs 3611 and 73D1 bind human C7 with a lower overall affinity compared to the above mAb but also bind rodent C7 proteins. The mAb 2H2 shows preferential binding to complexes making affinity measurements difficult.

In Vivo Complement Inhibition by mAb 17E7 and 73D1

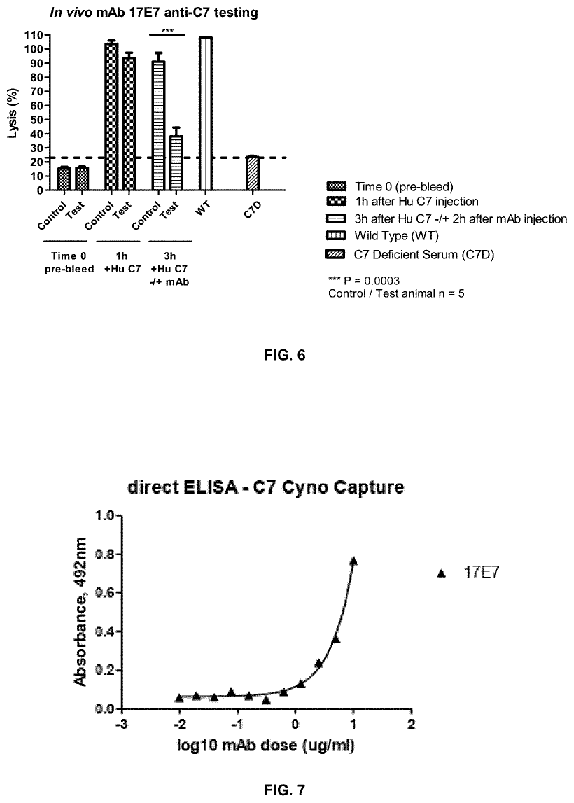

The results are shown in FIGS. 6 and 30.

Pre-bleed background haemolysis in C7-deficient mouse serum was <15%. One hour after administration of human C7, haemolytic activity in mice was markedly increased, indistinguishable from wild-type serum. Administration of mAb 17E7 (test) inhibited haemolysis (by about 70% at 2 hours post-administration) compared to irrelevant antibody (control), demonstrating that 17E7 efficiently blocked human C7 in vivo. 17E7 inhibits haemolytic activity in a mouse lacking mouse C7 that has been administered human C7 to restore complement activity.

This demonstrates that C7 inhibition in vivo using 17E7 inhibits systemic complement activity.

FIG. 30 shows that 73D1 efficiently inhibits complement activity in mice in vivo.

Wild type (WT) mice (C57BL/6J) were administered 73D1 or as a positive control the blocking anti-mouse C5 mAb BB5.1. WT mice were pre-bled and intraperitoneally injected with mAb 73D1 or BB5.1 (5 mice each). Complement activity was monitored by testing animal sera in the haemolytic assay over a time course of 48 hours using the add-back (to human depleted sera) assay. Complement was inhibited by both mAb over the full course of the experiment from 2-48 hours. BB5.1 has been widely used in animal models and set the scene for current anti-C5 therapeutics; 73D1 was administered at the same dose as BB5.1 and the efficient inhibition of mouse complement by this mAb make it a valuable candidate for animal studies targeting MAC specifically without interfering with C5a generation.

mAb 17E7 CDR Sequence Determination

The CDR sequences were determined by Absolute Antibody.

The results are summarized below.

Full VH Sequence
(SEQ ID NO: 1)
EVQLQQSGTVLARPGASVKMSCKTSGYSFTTYWMHWVKQRPGQGLEWIG
AIYPGNSDTYYSQKFKDKAKLTAVTSASTAYMELSSLTNEDSAVYYCTR
SGGYDVGGLDYWGQGTTLTVSS
CDR-H1
(SEQ ID NO: 2)
TYWMH
CDR-H2
(SEQ ID NO: 3)
AIYPGNSDTYYSQKFKD
CDR-H3
(SEQ ID NO: 4)
SGGYDVGGLDY
Full VL Sequence
(SEQ ID NO: 5)
DIVMTQSPATLSVTPGDGVSLSCRASQSISDYLHWYQQKSHESPRLLIK
FASQSISGIPSRFSGSGSGSDFTLSINSVEPEDVGMYYCQNGHSFPFTF
GSGTKLELK
CDR-L1
(SEQ ID NO: 6)
RASQSISDYLH
CDR-L2
(SEQ ID NO: 7)
FASQSIS
CDR-L3
(SEQ ID NO: 8)
QNGHSFPFT
mAb 2H2 CDR Sequence Determination
The CDR sequences were determined by Absolute
Antibody.
The results are summarized below.
Full VH Sequence
(SEQ ID NO: 11)
QVQLQQPGAELMKPGASVKLSCKASGYIFTSYWMHWVKQRPGRGLEWIG
RIDPNSGGTNYNEKFKSKATLTVDKPSSTVYMKISSLTSEDSAVYYCAR
EHDYDYAMDYWGQGTSVTVSS
CDR-H1
(SEQ ID NO: 12)
SYWMH
CDR-H2
(SEQ ID NO: 13)
RIDPNSGGTNYNEKFKS
CDR-H3
(SEQ ID NO: 14)
EHDYDYAMDY
Full VL Sequence
(SEQ ID NO: 15)
DIVMTQAAPSVPVTPGESVSISCRSSKSLLHSNGNTYLYWFLQRPGQSP
QLLIYRMSNLASGVPDRFSGSGSGTAFTLRISRVEAEDVGVYYCMQHLE
YPFTFGSGTKLEIK
CDR-L1
(SEQ ID NO: 16)
RSSKSLLHSNGNTYLY
CDR-L2
(SEQ ID NO: 17)
RMSNLAS
CDR-L3
(SEQ ID NO: 18)
MQHLEYPFT
mAb 73D1 CDR Sequence Determination
The CDR sequences were determined by Absolute
Antibody.
The results are summarized below.
Full VH Sequence
(SEQ ID NO: 19)
EVQLQQSGPELVKPGASVKMSCKASGYTFTDYNMHWVKQSHGKSLEWI
GYINPNNGGPSYNQNFKGKATLTVNKSSSTAYMELRSLTSEDSAVYYC
ACGNYPDYWGQGTTLTVSS
CDR-H1
(SEQ ID NO: 20)
DYNMH
CDR-H2
(SEQ ID NO: 21)
YINPNNGGPSYNQNFKG
CDR-H3
(SEQ ID NO: 22)
GNYPDY
Full VL Sequence
(SEQ ID NO: 23)
DIQMTQSPASLSASVGETVTITCRASGNIHNYLAWYQQKQGKSPQLLV
YNAKTLADGVPSRFSGSGSGTQYSLKINSLQPEDFGSYYCQHFWSTPL
TFGAGTKLELK
CDR-L1
(SEQ ID NO: 24)
RASGNIHNYLA
CDR-L2
(SEQ ID NO: 25)
NAKTLAD
CDR-L3
(SEQ ID NO: 26)
QHFWSTPLT

Direct Enzyme Linked Immunosorbent Assay (ELISA) to Test Cross-Reactivity

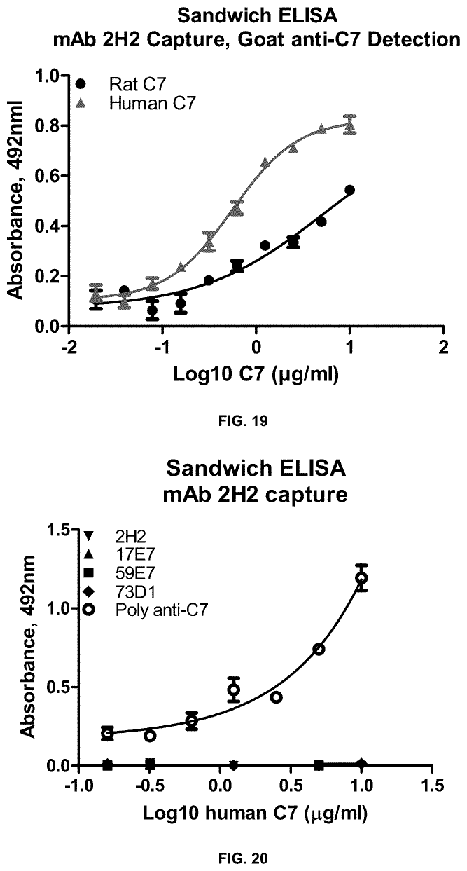

The results are shown in FIGS. 7 and 16 to 19. It was found that all antibodies including 17E7 bind strongly to cynomolgus monkey C7. 2H2 and 73D1 bind rat C7. 73D1 binds mouse C7.

Dot Blot to Test Binding of C7-FIMs by the Selected Anti-C7 mAb

The results indicate that 17E7 binds the C7 FIMs.

Sandwich ELISA to Test Epitope Competition for Binding to Human C7

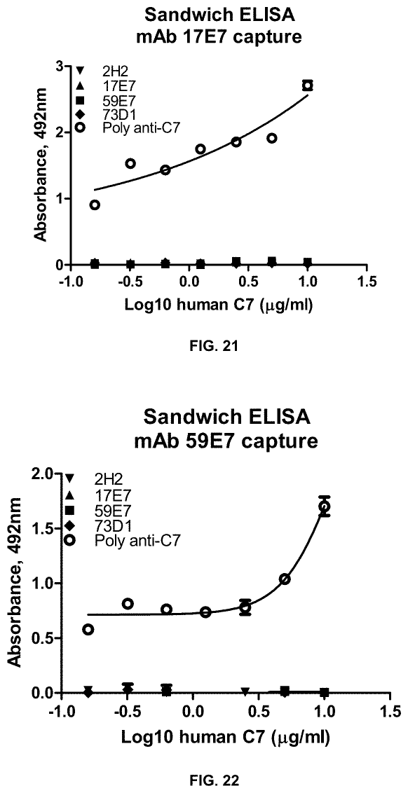

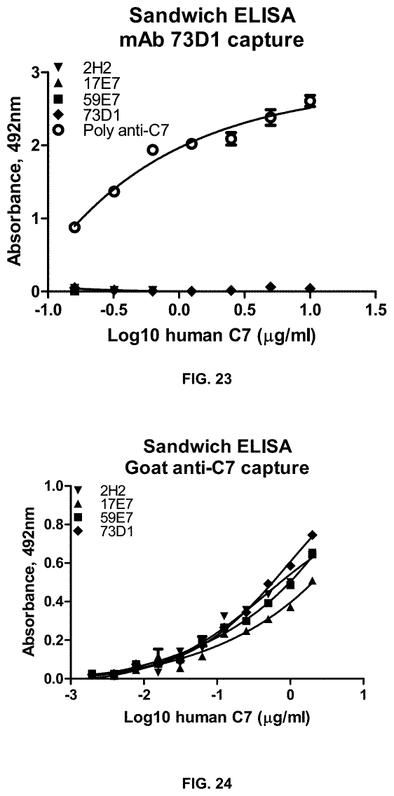

The results are shown in FIGS. 20 to 24. It was found that no combination of the four inhibitory antibodies work as pairs in ELISA. This suggests that they bind to similar epitopes on C7. Polyclonal goat anti-C7 antibody (Poly) recognising multiple epitopes of the C7 protein was used as the control.

Conclusions

The human specific monoclonal anti-C7 antibody (17E7) is an efficient inhibitor of complement in vitro and in vivo (tested in C7-deficient mice administered human C7). The mAb inhibited complement in human serum at similar levels to anti-C5 mAb

Eculizumab and SKY59 used as positive controls.

### Example 2

Preparation of Antibodies

Monoclonal antibodies against C7 protein were generated by first establishing a line of C7 deficient mice. CRISP-generated heterozygous C7 knock-out mice (C57BL/6NJ-C7^{em1(IMPC)J}/Mmjax) were purchased from Jackson Laboratory and back-crossed to obtain homogenous C7 deficient mice. These mice were immunized with rat C7 and human C7 (both purified in-house). The C7 deficient mice were also used as a source of macrophages during the cloning process as a feeding support for hybridoma cell line. The anti-C7 antibody 2H2 was selected for further testing. The hybridoma that produces 2H2 was deposited under the ECACC accession number 19080603 on 6 Aug. 2019.

Monoclonal anti-C5 antibodies were also generated in-house by the same methods but using C5-deficient mice immunised with pure C5 to use as controls. The control antibodies used were as follows.

7D4: Human/rat specificity

BB5.1: Mouse specificity

Test Methods

The same test methods described above in relation to Example 1 were also used in Example 2.

Dose-Dependent Complement Inhibition In Vivo in Rats and Suppress Disease in a Myasthenia Model

To test in vivo effects and therapeutic efficacy of test mAb, Lewis rats (100-150 g, Charles River Laboratories, Edinburgh, UK) were allowed to acclimatize for one week prior to disease induction by intraperitoneal administration of anti-Acetylcholine receptor (AChR) mAb35 at 1 mg/kg in PBS as described previously (Chamberlain-Banoub et al 2006). MAb35 binds the main immunogenic region of AChR, activating complement and damaging the neuromuscular junction endplates, causing severe muscle weakness. Animals were assessed hourly post-disease initiation (Clinical Score; 1). Reduced grip strength in front legs and floppy tail, 2). loss of grip in front legs, 3). loss of grip and hind limb weakness and wasting, 4). loss of grip and hind limb paralysis, 5). Moribund). mAb35-injected rats were split into two groups: Group one (n=5) was treated with test mAb 2H2 at lower dose of mAb (10 mg/kg; determined in a pilot study) subcutaneously (SC) at time zero. The second group (n=5) received an irrelevant isotype control (D1.3) antibody at the same times, routes and doses. All animals were euthanised at 48 hours' post-induction, blood taken for serum assays, and soleus muscles harvested and frozen in OCT mounting medium for sectioning as 10 μm and used for immunofluorescence to quantify AChR-positive endplates and to determine deposition of complement C3b/iC3b and C9 proteins.

In Vivo Testing of 2H2 in an EAMG Model—Soleus Staining to Determine Endplates Damage and Complement Proteins Deposition

Soleus sections were fixed in ice cold acetone for 15 min at −80° C. and then blocked for 30 minutes in 10% horse serum 2% BSA. After washing in PBS, sections were stained overnight at 4° C. with primary antibodies, C3/30 anti-C3b/iC3b mAb (in house) at 10 μg/ml, and rabbit anti-rat C9/MAC polyclonal IgG (in house) at 50 μg/ml, both in the block buffer. Anti-C3b/iC3b sections were washed and incubated for 15 min at RT with amplifier antibody goat anti-mouse (Vector Labs, Peterborough, UK, VectaFluor DyLight 488, #DK-2488). After washing, secondary antibody, horse anti-goat IgG-Alexa Fluor 488 (DyLight 488, #DK-2488) for C3b/iC3b or goat anti-rabbit-FITC (Oxford Biomedical Research, #45002) for anti-C9/MAC were added as appropriate, together with alpha-bungarotoxin-TRITC (BtX) (labels AChR; Boitum, #00012) at 0.5% and Hoechst stain 1:10 000 (ThermoFisher, #62249), then incubated 40 minutes at RT in dark. Sections were washed in PBS and mounted in VectorShield Vibrance (Vector labs, #H-1700-2) before analysis using an Apotome fluorescent microscope (Zeiss Apotome Axio Observer microscope). Slides were prepared from each test and control group animal to obtain at least ten fields captured from comparable regions of muscle in each sample at the same exposure and magnification (×40). The number of BuTx-reactive endplates in each section was measured using density slicing in an image analysis system (ImageJ). Appropriate controls; secondary antibody only, primary only and no staining visualised.

Results

Direct Enzyme Linked Immunosorbent Assay and Sandwich Enzyme Linked Immunosorbent Assay (ELISA)

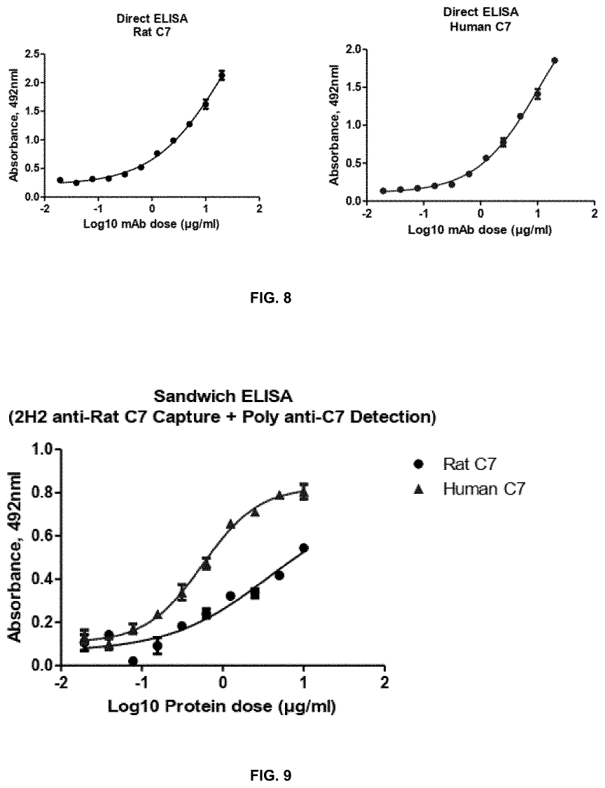

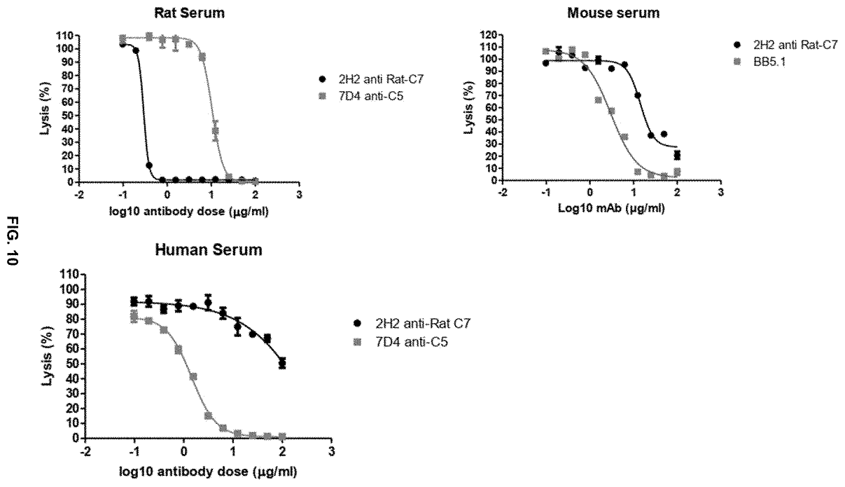

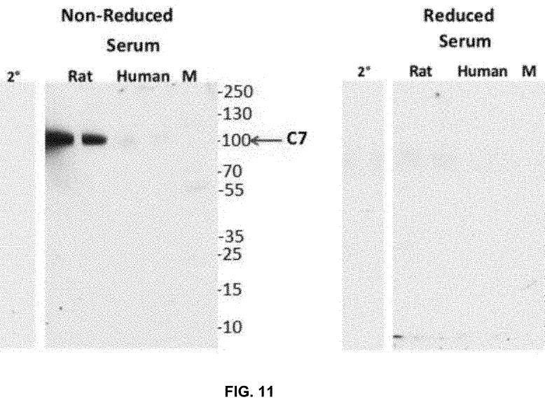

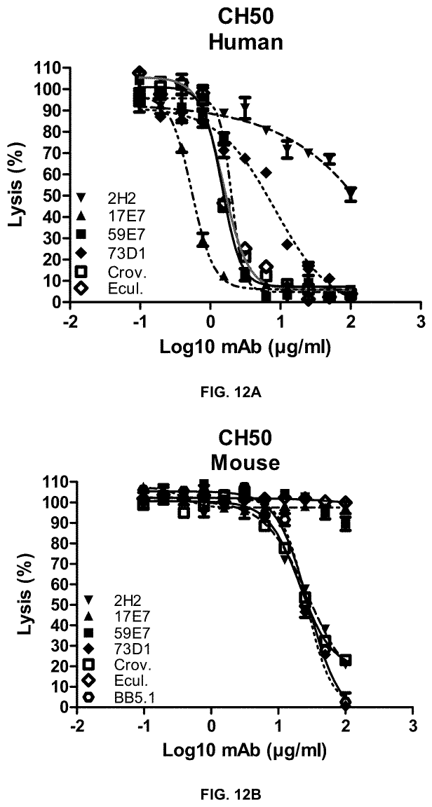

The results are shown in FIGS. 8, 9, 16, 17 and 19. It was found that clone 2H2 binds rat and human C7 in direct ELISA and sandwich ELISA. 2H2 also binds rat C7.

Standard Classical Pathway Haemolysis Assays (CP)

Assay Protocol: Standard classical pathway haemolysis assays (CP50)

mAb dilution: 100/50/25/12.5/6.3/3.1/1.6/0.8/0.39/0.20/0.10/0 μg/ml

Serum dilution: 2.5

Erythrocytes: 2% ShEA

The results are shown in FIGS. 10 and 12, and Table 2 above.

It was found that 2H2 is an efficient inhibitor of rat C7. A much lower dose is required for 50% inhibition of lysis compared to the dose required for 50% inhibition of lysis of C5 for either of the comparator anti-C5 antibodies.

Serum Excess Classical Pathway Haemolysis Assays (Serum Excess CP)

Assay Protocol: Serum excess classical pathway haemolysis assays (Serum Excess CP)

mAb dilution: 1000/500/250/125/63/31/16/8/3.9/2/1/0 μg/ml

Human, rat, serum dilution: 2.5%; rabbit, mouse: 25%

Erythrocytes: 2% ShEA

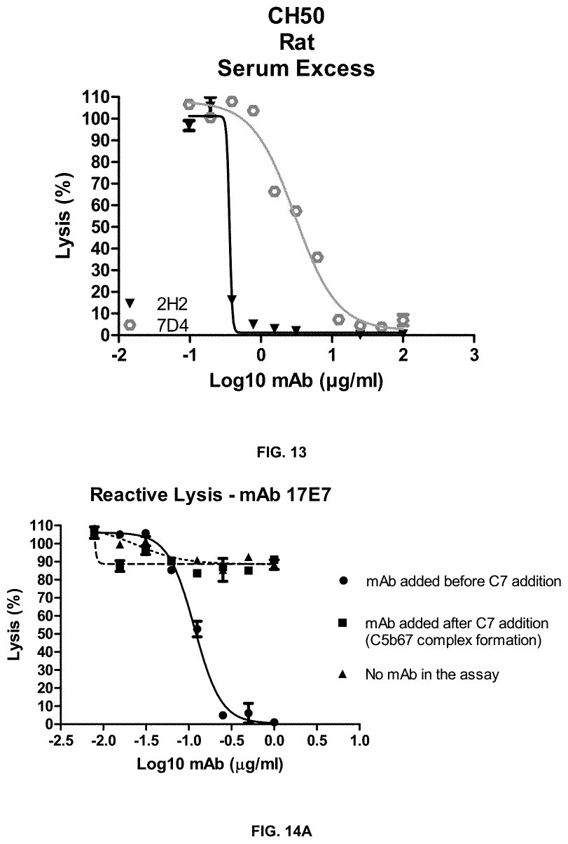

The results are shown in FIG. 13.

It was found that 2H2 inhibited lysis in this assay.

Western Blot

The results are shown in FIGS. 11 and 25. It was found that 2H2 strongly detects rat C7 protein, and weakly human C7 protein, in serum under non-reducing but not reducing conditions. 2H2 also binds rat, mouse (Ms) and monkey (Mk) C7. In addition to C7, 2H2 also binds high molecular weight aggregates in serum.

Western Blot and ELISA on mAb Pull-Down from Activated Serum to Identify the Composition of the Pull-Down Complex

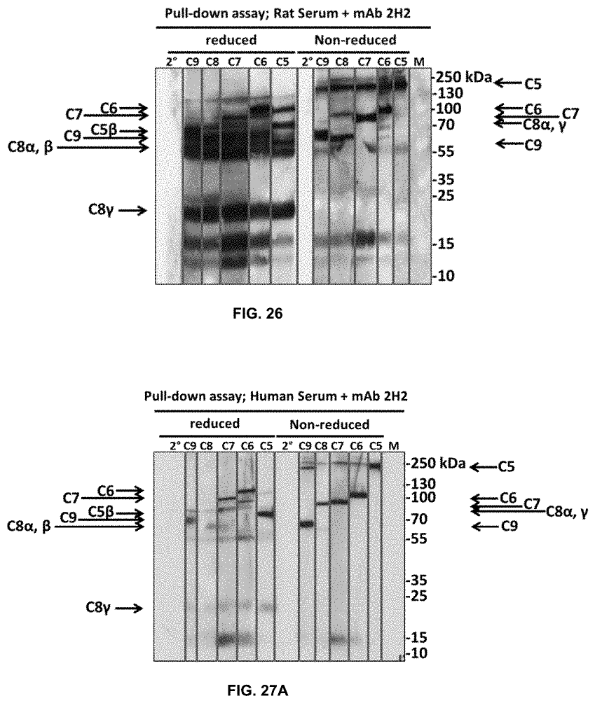

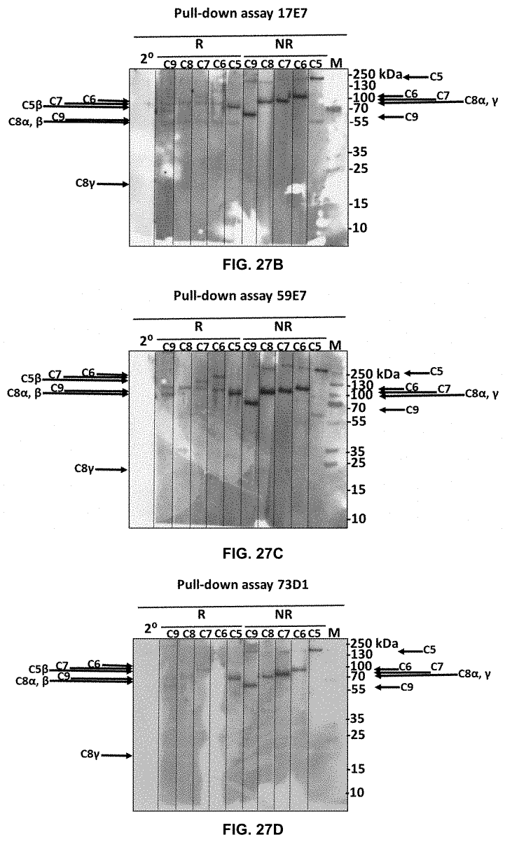

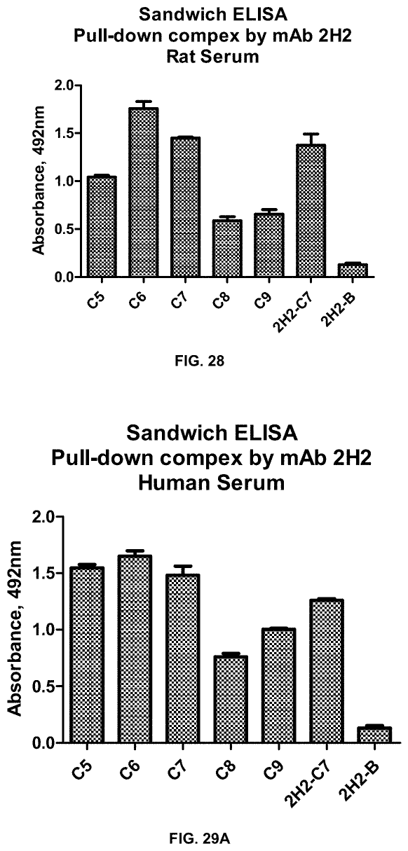

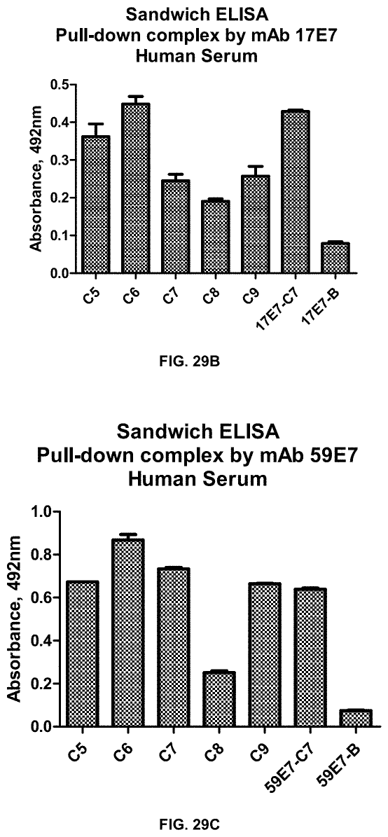

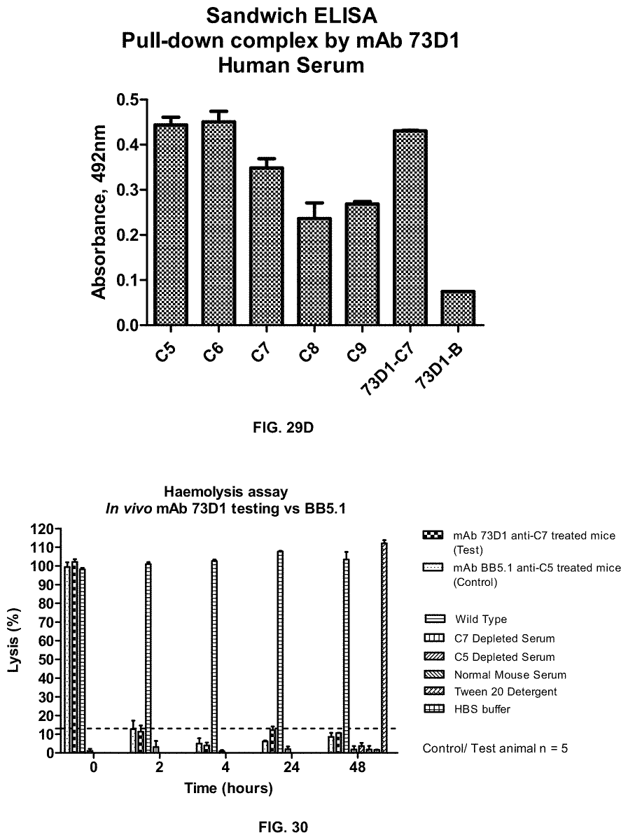

The results are shown in FIGS. 26 to 29. It was found that each of the mAbs 2H2, 17E7, 59E7 and 73D1 bound and pulled-down the C5b-9 complex from activated serum

Rat Cross-Reactive mAb 2H2 Shows Dose-Dependent Complement Inhibition In Vivo in Rats and Suppress Disease in the Experimental Autoimmune Myasthenia Gravis (EAMG) Model

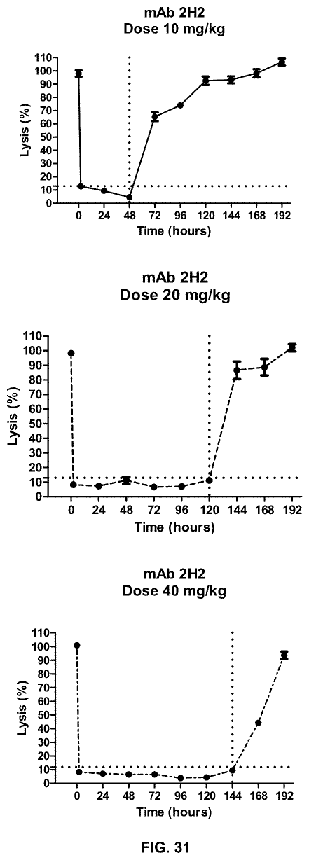

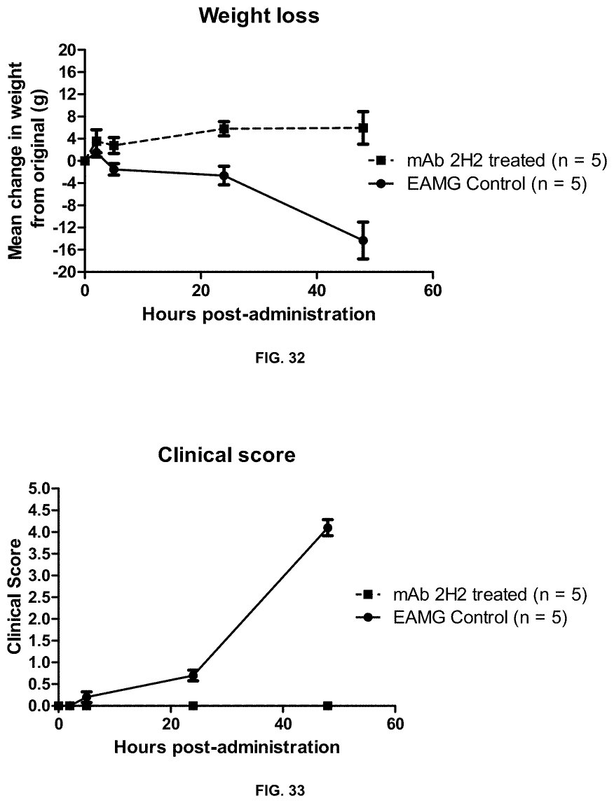

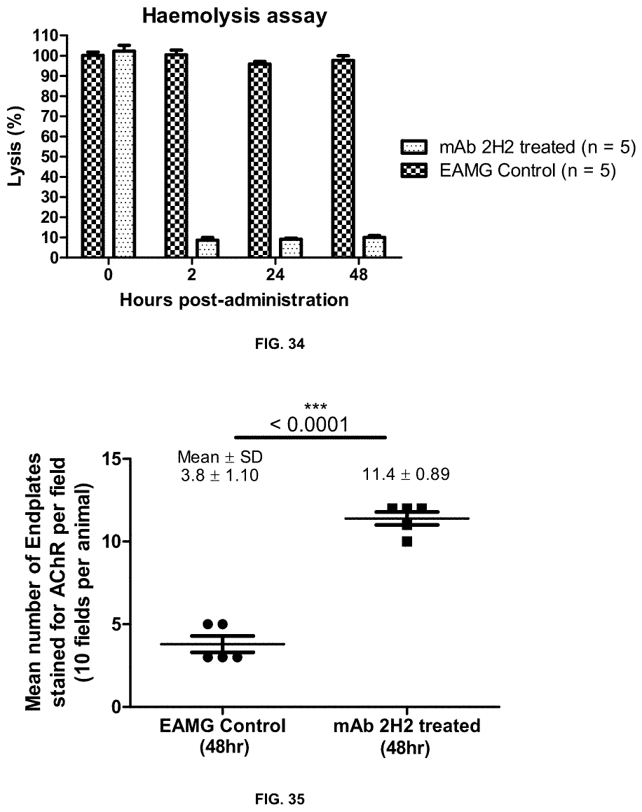

The results are shown in FIGS. 31 to 34. It was found that low doses of 2H2 (10 mg/kg) efficiently inhibit complement and protect from Myasthenic disease in rats. Rats treated with 2H2 showed no symptoms of disease and no weight loss.

In Vivo Testing of 2H2 in an EAMG Model—Soleus Muscle Staining to Determine Endplate Damage and Complement Protein Deposition

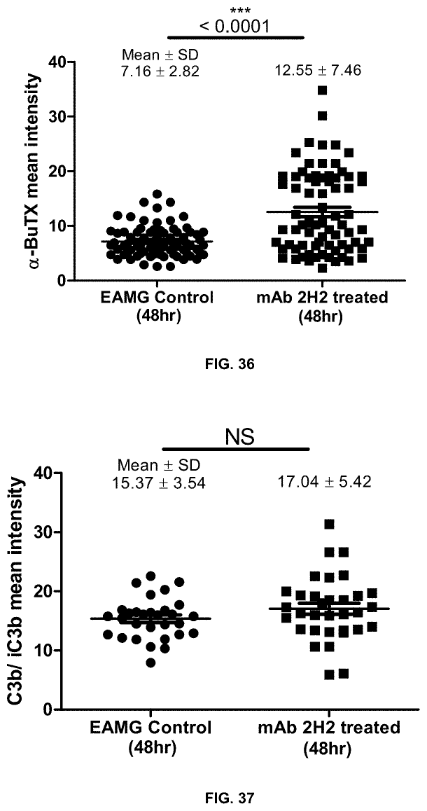

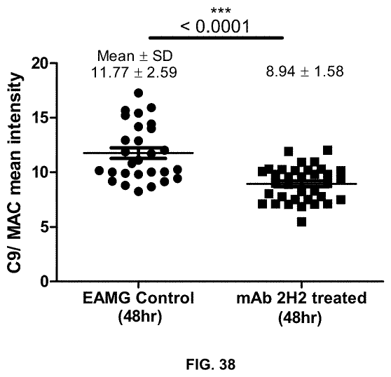

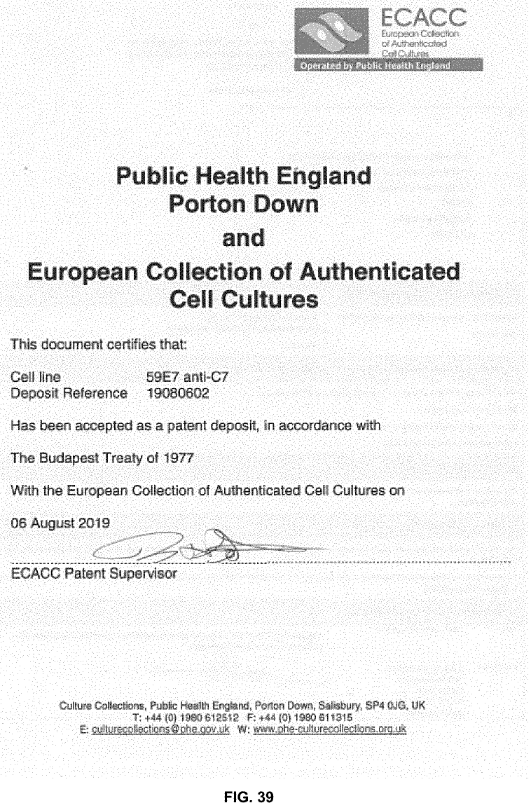

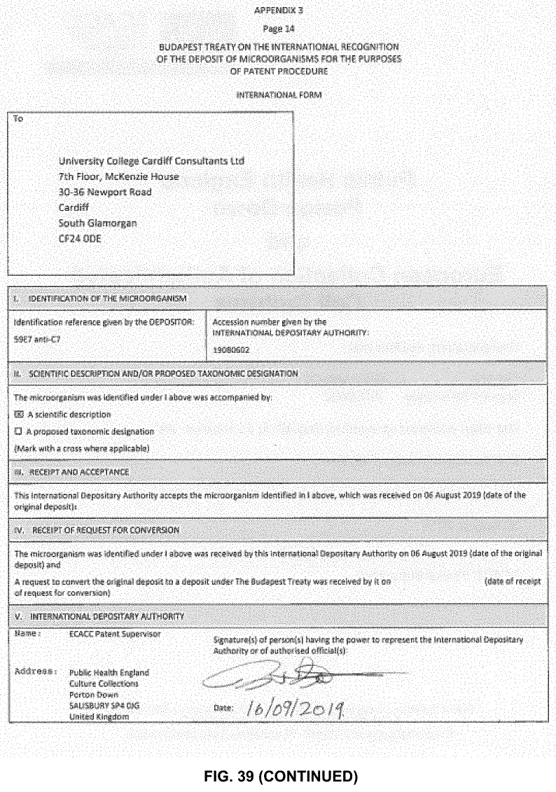

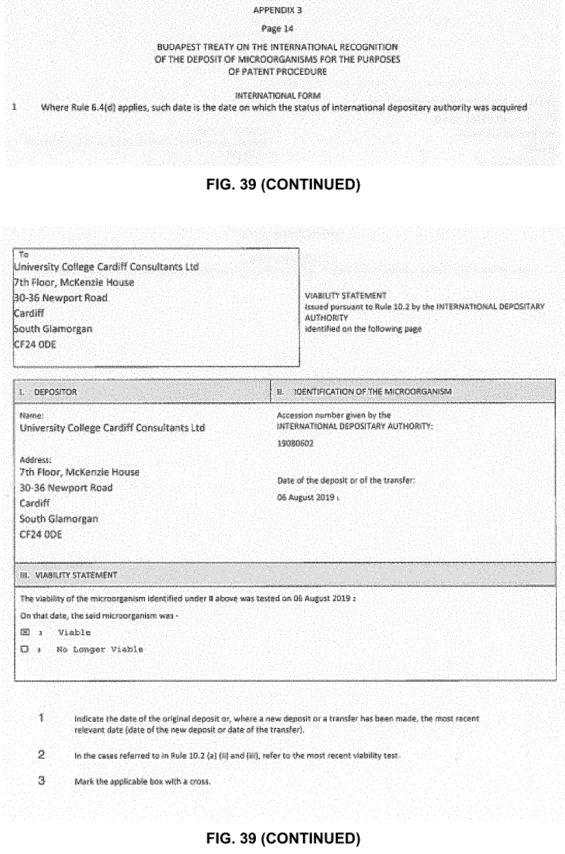

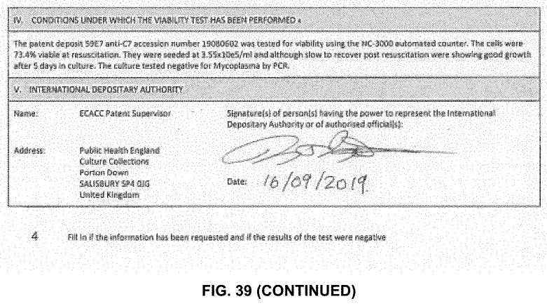

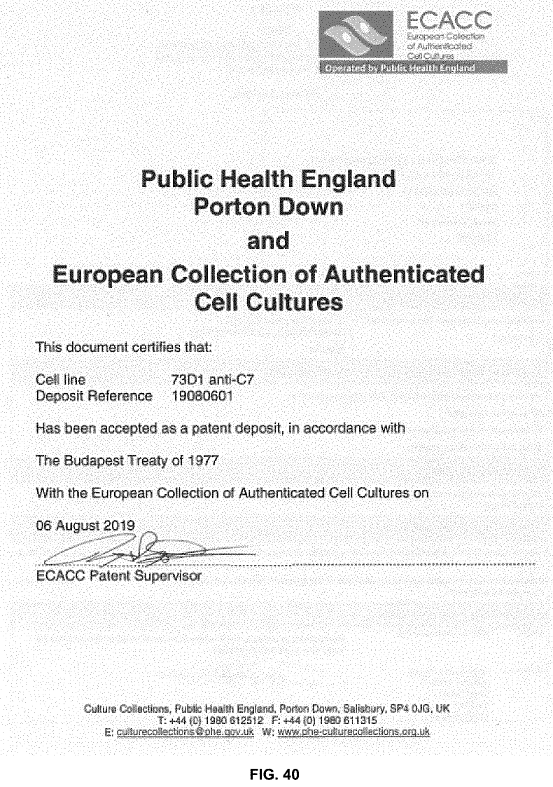

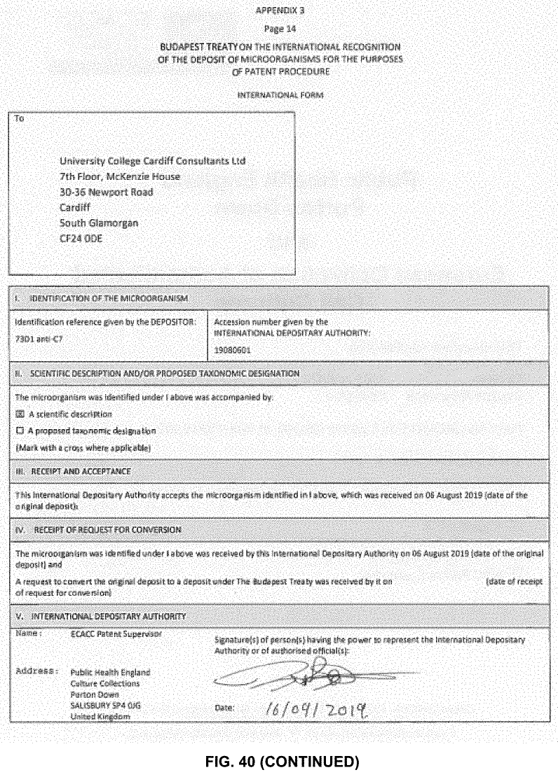

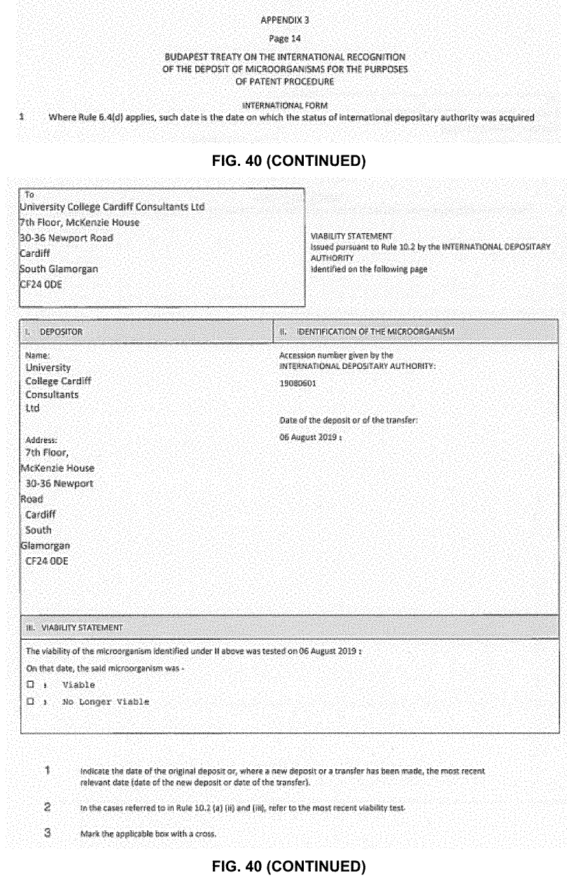

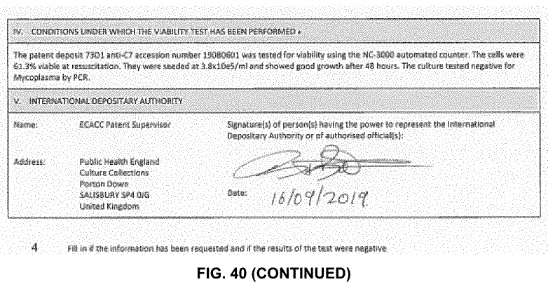

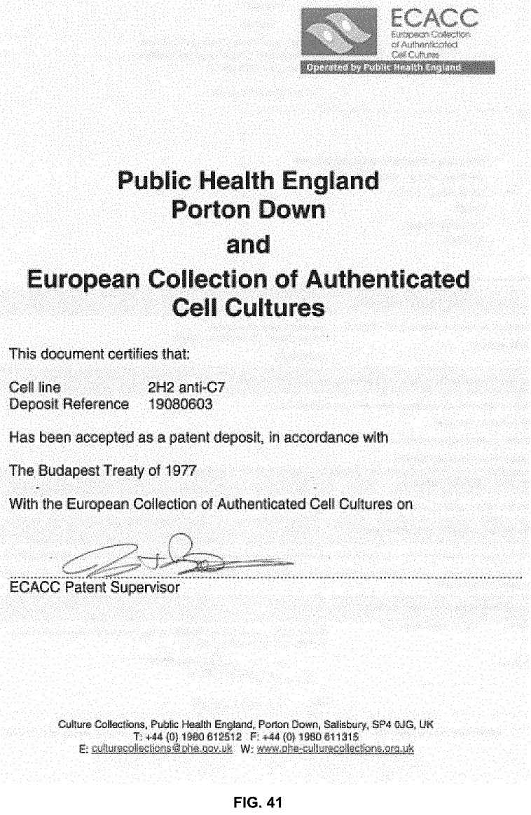

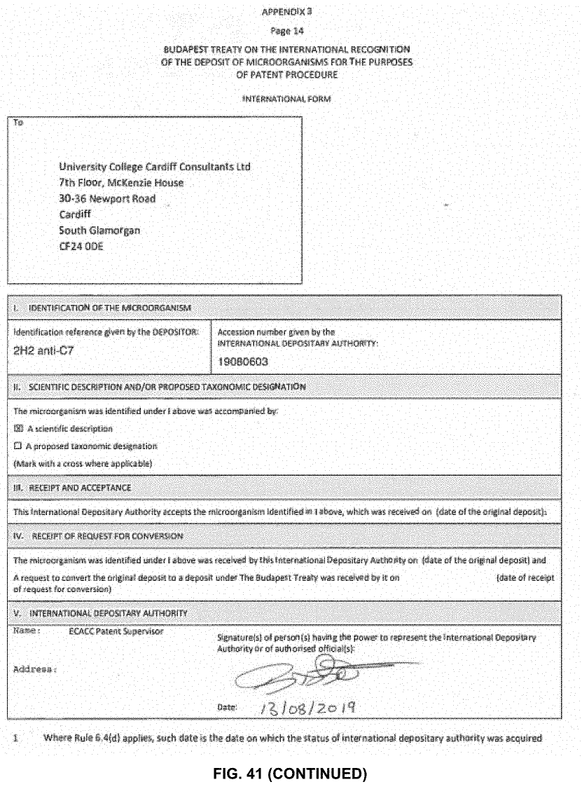

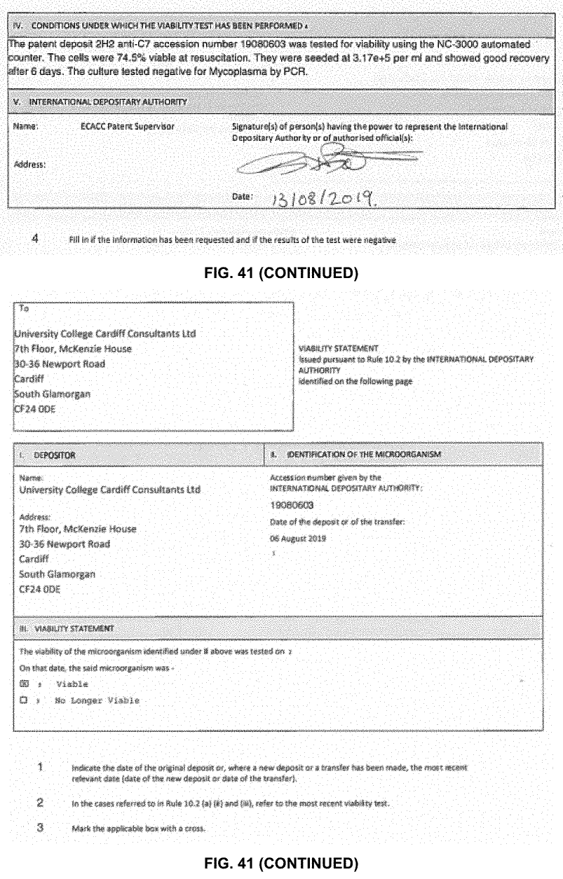

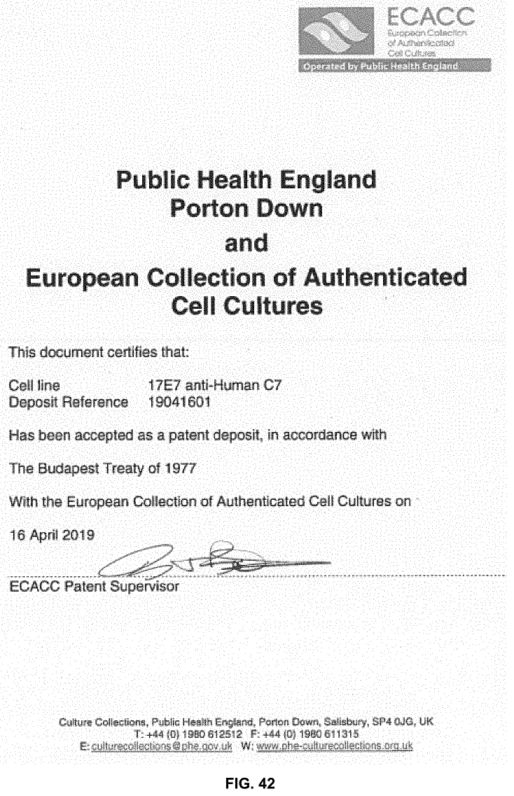

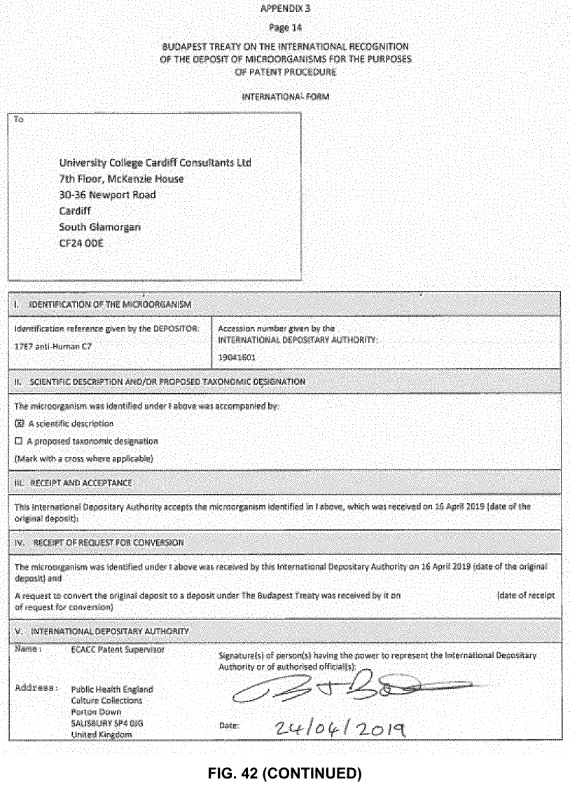

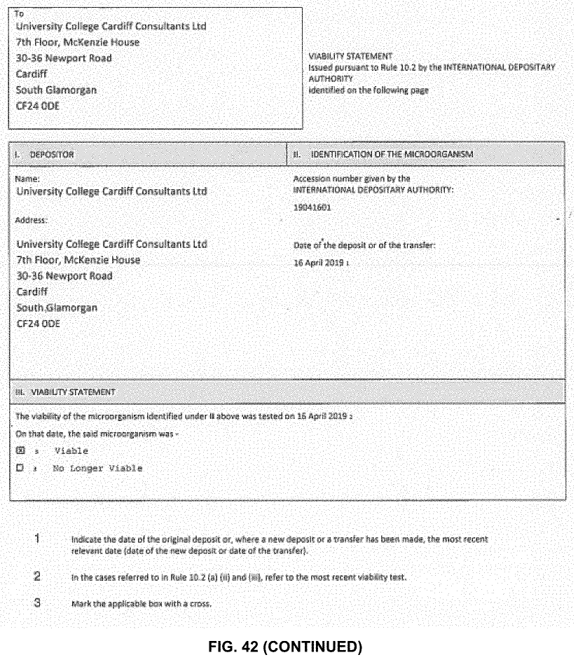

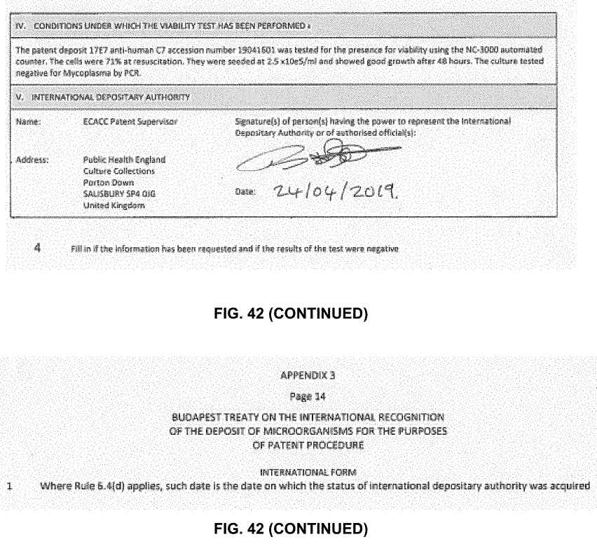

The results are shown in FIGS. 35 to 38. It was found that endplates in the Myasthenia model were reduced in number, fragmented and richly decorated with C3b/iC3b and C9/MAC, whereas in 2H2-treated animals endplates were preserved in number and integrity. Although endplates were strongly C3b/iC3b-positive in these animals, C9/MAC deposition was markedly reduced, reflecting terminal pathway blockade.

### CONCLUSIONS

Each of the mAb bound the C5b-9 complex in activated serum. The monoclonal anti-C7 antibody 2H2 is an efficient inhibitor of complement in vitro and in vivo and protects from complement-driven pathology in a disease model.

The foregoing broadly describes certain embodiments of the present invention without limitation. Variations and modifications as will be readily apparent to those skilled in the art are intended to be within the scope of the present invention as defined in and by the appended claims.

1. An antibody or an antibody fragment that binds to the complement protein C7 in isolation or as part of a protein complex and inhibits complement activation.

2. The antibody or antibody fragment of claim 1, wherein the complement protein C7 is human complement protein C7.

3. The antibody or antibody fragment of claim 1, wherein the antibody or antibody fragment binds to one or both of the Factor 1-like modules (FIMs) of the complement protein C7.

4. The antibody or antibody fragment of claim 1, wherein the antibody or antibody fragment inhibits binding of the complement protein C7 to the complement protein complex C5b6 and/or binds C7 as part of the C5b67 complex preventing its insertion into the membrane and/or wherein the antibody or antibody fragment inhibits binding of the complement protein C8 to the complement protein complex C5b67.

5. The antibody or antibody fragment of claim 1, wherein the antibody fragment comprises at least one heavy chain variable domain and/or at least one light chain variable domain, for example wherein the antibody fragment comprises two heavy chain variable domains and two light chain variable domains.

6. The antibody or antibody fragment of claim 1, wherein the antibody or antibody fragment has a binding affinity constant (KD) for C7 in isolation or as part of a protein complex equal to or less than about 5×107 M; and/or
a binding association rate (ka) for C7 in isolation or as part of a protein complex equal to or greater than about 2.5×103 M−1 s−1; and/or
a binding dissociation rate (kd) for C7 in isolation or as part of a protein complex equal to or less than about 5×10−3 s−1.

7.-8. (canceled)

9. The antibody or antibody fragment of claim 1, wherein the antibody or antibody fragment comprises:
an amino acid sequence having at least about 60% homology to SEQ ID NO: 2 or 12 or 20; and/or
an amino acid sequence having at least about 60% homology to SEQ ID NO: 3 or 13 or 21; and/or
an amino acid sequence having at least about 60% homology to SEQ ID NO: 4 or 14 or 22.

10. The antibody or antibody fragment of claim 1, wherein the antibody or antibody fragment comprises:
an amino acid sequence having up to 3 amino acid differences to SEQ ID NO: 2 or 12 or 20; and/or
an amino acid sequence having up to 3 amino acid differences to SEQ ID NO: 3 or 13 or 21; and/or
an amino acid sequence having up to 3 amino acid differences to SEQ ID NO: 4 or 14 or 22.

11. The antibody or antibody fragment of claim 1, wherein the antibody or antibody fragment comprises:
an amino acid sequence having at least about 60% homology to SEQ ID NO: 6 or 16 or 24; and/or
an amino acid sequence having at least about 60% homology to SEQ ID NO: 7 or 17 or 25; and/or
an amino acid sequence having at least about 60% homology to SEQ ID NO: 8 or 18 or 26.

12. The antibody or antibody fragment of claim 1, wherein the antibody or antibody fragment comprises:
an amino acid sequence having up to 3 amino acid differences to SEQ ID NO: 6 or 16 or 24; and/or
an amino acid sequence having up to 3 amino acid differences to SEQ ID NO: 7 or 17 or 25; and/or
an amino acid sequence having up to 3 amino acid differences to SEQ ID NO: 8 or 18 or 26.

13. The antibody or antibody fragment of claim 1, wherein the antibody or antibody fragment comprises an amino acid sequence having at least about 60% homology to SEQ ID NO: 1 or 11 or 19 and/or an amino acid sequence having at least about 60% homology to SEQ ID NO: 5 or 15 or 23.

14. (canceled)

15. The antibody or antibody fragment of claim 1, wherein the antibody is obtained from or obtainable from the hybridoma having the ECACC accession number 19041601 or the hybridoma having the ECACC accession number 19080601 or the hybridoma having the ECACC accession number 19080602 or the hybridoma having the ECACC accession number 19080603.

16. The antibody or antibody fragment of claim 1, wherein the antibody or antibody fragment is a humanized antibody or antibody fragment, or a chimeric antibody or antibody fragment, or an affinity-matured antibody or antibody fragment, or a recombinant antibody or antibody fragment.

17. A pharmaceutical composition comprising an antibody or antibody fragment of claim and a pharmaceutically acceptable excipient, diluent, or carrier.

18. A nucleic acid sequence encoding the antibody or antibody fragment of claim 1.

19. A cell expressing the nucleic acid sequence of claim 18 or a cell secreting an antibody or antibody fragment of any of claims 1 to 16.

20. The cell of claim 19 wherein the cell is a hybridoma, for example having the ECACC accession number 19041601 or the ECACC accession number 19080601 the ECACC accession number 19080602 the ECACC accession number 19080603.

21. (canceled)

22. A method of treating or preventing a disease associated with the dysregulation of complement in a subject, the method comprising administering the antibody or antibody fragment or pharmaceutical composition or nucleic acid sequence of claim to the subject.

23. A non-therapeutic use of an antibody or antibody fragment of claim 1 for detecting C7, for example for detecting C7 in a method of diagnosing or monitoring a disease.

24. The method of claim 23, wherein the disease is selected from the group consisting of paroxysmal nocturnal hemoglobinuria (PNH), atypical haemolytic uremic syndrome (aHUS), hereditary angioedema (HAE), mismatched blood transfusions, blood compatibility diseases (e.g. erythroblastosis fetalis), systemic lupus erythematosus (SLE), C3 glomerulopathy (C3G), age-related macular degeneration (AMD), myasthenia gravis, neuromyelitis optica, multiple sclerosis (MS), rheumatoid arthritis (RA), Hidradenitis suppuritiva, polyarteritis, inflammatory effects of cardiopulmonary bypass and haemodialysis, transplant rejection, (ANCA)-associated vasculitis, Alzheimer's disease, atypical haemolytic uremic syndrome, acute kidney injury, antibody-mediated rejection, antiphospholipid syndrome, acute respiratory distress syndrome, Berger's disease, bullous pemphigoid, cold agglutinin disease, chronic obstructive pulmonary disease, cardiopulmonary bypass, dense deposit disease, delayed graft function, geographic atrophy, Guillain-Barre syndrome, refractory generalized myasthenia gravis, granulomatosis with polyangiitis, graft versus host disease, hereditary angioedema, IgA nephropathy, hematopoietic stem cell transplant-related TMA, ischemia/reperfusion injury, immune complex membranoproliferative glomerulonephritis, idiopathic polypoidal choroidal vasculopathy, kidney transplant, lupus nephritis, membranous nephropathy, microscopic polyangiitis, neuromyelitis optical spectrum disorder, rheumatoid arthritis/osteoarthritis, systemic inflammatory response syndrome, systemic lupus erythematosus, Stargardt disease 1, sepsis, severe sepsis, septic shock, thrombotic microangiopathy, warm type autoimmune haemolytic anemia, wet AMD, periodontitis, asthma, Crohn's disease, atherosclerosis, vasculitis, hemodialysis, psoriasis, burns, myocardial infarction, glaucoma, uveitis, trauma, Alzheimer's disease, and Parkinson's disease.
